SEQUENCE LISTING (95 sequences)

SEQ ID NO: 1 - DNA, 2045 bp, Homo sapiens
  gene (1)..(2045): The sequence of the cDNA coding for 1-acylglycerol-3-phosphate acyltransferase
cgacaccccgacagagacagagacacagccatccgccaccaccgctgccgcagcctggctggggagggggccagccccccaggccccctacccctctgaggtggccagaatggatttgtggccaggggcatggatgctgctgctgctgctcttcctgctgctgctcttcctgctgcccaccctgtggttctgcagccccagtgccaagtacttcttcaagatggccttctacaatggctggatcctcttcctggctgtgctcgccatccctgtgtgtgccgtgcgaggacgcaacgtcgagaacatgaagatcttgcgtctaatgctgctccacatcaaatacctgtacgggatccgagtggaggtgcgaggggctcaccacttccctccctcgcagccctatgttgttgtctccaaccaccagagctctctcgatctgcttgggatgatggaggtactgccaggccgctgtgtgcccattgccaagcgcgagctactgtgggctggctctgccgggctggcctgctggctggcaggagtcatcttcatcgaccggaagcgcacgggggatgccatcagtgtcatgtctgaggtcgcccagaccctgctcacccaggacgtgagggtctgggtgtttcctgagggaacgagaaaccacaatggctccatgctgcccttcaaacgtggcgccttccatcttgcagtgcaggcccaggttcccattgtccccatagtcatgtcctcctaccaagacttctactgcaagaaggagcgtcgcttcacctcgggacaatgtcaggtgcgggtgctgcccccagtgcccacggaagggctgacaccagatgacgtcccagctctggctgacagagtccggcactccatgctcactgttttccgggaaatctccactgatggccggggtggtggtgactatctgaagaagcctgggggcggtgggtgaaccctggctctgagctctcctcccatctgtccccatcttcctccccacacctacccacccagtgggccctgaagcagggccaaaccctcttccttgtctcccctctccccacttattctcctctttggaatcttcaacttctgaagtgaatgtggatacagcgccactcctgccccctcttggccccatccatggactcttgcctcggtgcagtttccactcttgacccccacctcctactgtcttgtctgtgggacagttgcctccccctcatctccagtgactcagcctacacaagggaggggaacattccatccccagtggagtctcttcctatgtggtcttctctacccctctaccccacattggccagtggactcatccattctttggaacaaatcccccccactccaaagtccatggattcaatggactcatccatttgtgaggaggacttctcgccctctggctggaagctgatacctgaagcactcccaggctcatcctgggagctttcctcagcaccttcaccttccctcccagtgtagcctcctgtcagtgggggctggacccttctaattcagaggtctcatgcctgcccttgcccagatgcccagggtcgtgcactctctgggataccagttcagtctccacatttctggttttctgtccccatagtacagttcttcagtggacatgaccccacccagccccctgcagccctgctgcaccatctcaccagacacaaggggaagaagcagacatcaggtgctgcactcacttctgccccctggggagttggggaaaggaacgaaccctggctggaggggataggagggcttttaatttatttctttttctgttgaggcttccccctctctgagccagttttcatttcttcctggtggcattagccactccctgcctctcactccagacctgttcccacaactggggaggtaggctgggagcaaaaggagagggtgggacccagttttgcgtggttggtttttattaattatctggataacagcaaaaaaactgaaaataaagagagagagagaaaaaaaaaaaaaaaaaaaaa

SEQ ID NO: 2 - DNA, 1554 bp, Homo sapiens
  gene (1)..(1554): The sequence of the cDNA coding for Aldehyde dehydrogenase (5 family, member A1)
atgctgcgcttcctggcaccccggctgcttagcctccagggcaggaccgccctctactcctcggcagcagccctcccaagccccattctgaacccagacatcccctacaaccagctgttcatcaacaatgaatggcaagatgcagtcagcaagaagaccttcccgacggtcaaccctaccaccggggaggtcatcgggcacgtggctgaaggtgaccgggctgatgtggatcgggccgtgaaagcagcccgggaagccttccgcctggggtccccatggcgccggatggatgcctctgagcggggccggctgctgaacctcctggcagacctagtggagcgggatcgagtctacttggcctcactcgagaccttggacaatgggaagcctttccaagagtcttacgccttggacttggatgaggtcatcaaggtgtatcggtactttgctggctgggctgacaagtggcatggcaagaccatccccatgcatggccagcatttctgcttcacccggcatgagcccgttggtgtctgtggccagatcatcccgtggaacttccccttggtcatgcagggttggaaacttgccccggcactcgccacaggcaacactgtggttatgaaggtggcagagcagacccccctctctgccctgtatttggcctccctcatcaaggaggcaggctttccccctggggtggtgaacatcatcacggggtatggcccaacagcaggtgcggccatcgcccagcacatggatgttgacaaagttgccttcaccggttccaccgaggtgggccacctgatccagaaagcagctggcgattccaacctcaagagagtcaccctggagctgggtggtaagagccccagcatcgtgctggccgatgctgacatggagcatgccgtggagcagtgccacgaagccctgttcttcaacatgggccagtgctgctgtgctggctcccggaccttcgtggaagaatccatctacaatgagtttctcgagagaaccgtggagaaagcaaagcagaggaaagtggggaacccctttgagctggacacccagcaggggcctcaggtggacaaggagcagtttgaacgagtcctaggctacatccagcttggccagaaggagggcgcaaaactcctctgtggcggagagcgtttcggggagcgtggtttcttcatcaagcctactgtctttggtggcgtgcaggatgacatgagaattgccaaagaggagatctttgggcctgtgcagcccctgttcaagttcaagaagattgaggaggtggttgagagggccaacaacaccaggtatggcctggctgcggctgtgttcacccgggatctggacaaggccatgtacttcacccaggcactccaggccgggaccgtgtgggtaaacacctacaacatcgtcacctgccacacgccatttggagggtttaaggaatctggaaacgggagggagctgggtgaggatgggcttaaggcctacacagaggtaaagacggtcaccatcaaggttcctcagaagaactcgtaa

SEQ ID NO: 3 - DNA, 2051 bp, Homo sapiens
  gene (1)..(2051): The sequence of the cDNA coding for Choline/ethanolamine phosphotransferase
ggcacgagctggagtcggaggcgatatttctaggggtgtacttgttggggtcagggtaagcaccagccacaaaaacctacaaaagaagggaaattactgtctttaaatattaaaaaaaaacaagatccatgagtgggcatcgatcaacaaggaaaagatgtggagattctcacccggagtccccagtgggcttcgggcatatgagtactacaggatgtgtattaaataaattgtttcagttaccaacaccaccattgtcaagacaccaactaaagcggctagaagaacacagatatcaaagtgctggacggtccctgcttgagcccttaatgcaagggtattgggaatggctcgttagaagagttccctcctggattgccccaaatctcatcaccatcattggactgtcaataaacatctgtacaactattttattagtcttctactgccctacagctacagagcaggcacctctgtgggcatatattgcttgtgcctgtggccttttcatttaccagtctttggatgctattgatgggaaacaggcaagaagaaccaatagtagttctcctctgggagaactttttgatcatggctgtgattcactatcaacagtttttgtggttcttggaacttgtattgcagtgcagctggggacaaaccctgattggatgtttttttgttgttttgcggggacatttatgttctattgtgcgcactggcaaacgtatgtttctggaacattgcgatttggaataattgatgtgactgaagtgcaaatcttcataataatcatgcatttgctggcagtgattggaggaccacctttttggcaatctatgattccagtgctgaatattcaaatgaaaatttttcctgcactttgtactgtagcagggaccatattttcctgtacaaattacttccgtgtaatcttcacaggtggtgttggcaaaaatggatcaacaatagcaggaacaagtgtcctttctccttttctccatattggatcagtgattacattagctgcaatgatctacaagaaatctgcagttcagctttttgaaaagcatccctgtctttatatactgacatttggttttgtgtctgctaaaatcactaataagcttgtggttgcacacatgacgaaaagtgaaatgcatttgcatgacacagcattcataggtccggcacttttgtttctggaccagtattttaacagctttattgatgaatatattgtactttggattgccctggttttctctttctttgatttgatccgctactgtgtcagtgtttgcaatcagattgcgtctcacctgcacatacatgtcttcagaatcaaggtctctacagctcattctaatcatcattaatgatgtaattggtatataggaacatcatgttttctgcaggaaagaaagtaacatattaaggagaatgggggtggataagaacaaatataatttataataatcaatgttgtataacttttattctttattattggtaacacgccctaactatcctgtgtgagaatgggaatttcaagtcccatcttgtaaattgtatatgttgtcatgcagggtttgggccaagaaagcatgcagaaaaaaatgccatgtgattgtaattatcctggattcagaataatactgtgatggggagccagatccgcagtggtggagagttctaatgttgactgtttgcaggccaaaagatgattgctttataattttaacaaatcattgtcttttagtaacatccttgtttagtgtcttctcaagctttctttactgaggaattcagcttgtgacacagatacatcccactagcttgtgaggtggaactagtaataaagaccttgaatttggattgaaaagtttcctatctttacattgttgaggaagtcctttttttttttttttttaattgctcaagaaatgattctctcacaggcttgggaaatcctgttagcatgcagaataatgtggtaactttgtcaatttcccattttatttttttaaataaatatatgatctaaacgg

SEQ ID NO: 4 - DNA, 3758 bp, Homo sapiens
  gene (1)..(3758): The sequence of the cDNA coding for Diacylglycerol kinase, gamma
cacggagatagacagctttggagctgctgaactccgagcacagggtgaagaccccggcgctaccaaccacagcctggcagcctggtctccgcggcacccactggggctgcatccccctcccccgagagggctgcgcaggcgggaagacgccagaggccagcttcggtcccccttctgtctctcggttcctctttcctcccaagtaagggaataaaccgcgaagaaggagcgccccgggccaccgcgcaaccaagtgttgcctggtgaggaagagccaggacttctgaatttaccttgaatacagacaggaggatgttgcctaaggaatagcagagatcttgtctcatcttctgagaggtgcctgctgctgctgtatacacttgagtgctcccagaagtctcctgaaaggcttacatcgcaaacctgcaatgagccaggccctgggctgggcctccacttcagcctagtgaacaaaactccatcactgccctttagccactcacataaagtttaaaaatgggtgaagaacggtgggtctccctcactccagaagaatttgaccaactccagaaatattcagaatattcctccaagaagataaaagatgccttgactgaatttaatgagggtgggagcctcaaacaatatgacccacatgagccgattagctatgatgtcttcaagctgttcatgagggcgtacctggaggtggaccttccccagccactgagcactcacctcttcctggccttcagccagaagcccagacacgagacctctgaccacccgacggagggagccagcaacagtgaggccaacagcgcagatactaatatacagaatgcagataatgccaccaaagcagacgaggcctgtgcccctgatactgaatcaaatatggctgagaagcaagcaccagctgaagaccaagtggctgcgacccccctggaaccccccgtccctcggtcttcaagctcggaatccccagtggtgtacctgaaggatgttgtgtgctacctgtccctgctggagacggggaggcctcaggataagctggagttcatgtttcgcctctatgattcagatgagaacggtctcctggaccaagcggagatggattgcattgtcaaccaaatgctgcatattgcccagtacctggagtgggatcccacagagctgaggcctatattgaaggagatgctgcaagggatggactacgaccgggacggctttgtgtctctacaggaatgggtccatggagggatgaccaccatcccattgctggtgctcctggggatggatgactctggctccaagggggatggggggcacgcctggaccatgaagcacttcaagaaaccaacctactgcaacttctgccatatcatgctcatgggcgtccgcaagcaaggcctgtgctgcacttactgtaaatacactgtccacgaacgctgtgtgtccaaaaacattcctggttgtgtcaaaacgtactcaaaagccaaaaggagtggtgaggtgatgcagcacgcatgggtggaagggaactcctccgtcaagtgtgaccggtgccacaaaagtatcaagtgctaccagagtgtcaccgcgcggcactgcgtgtggtgccggatgacgtttcaccgcaaatgtgaattatcaacgttgtgtgacggtggggaactcagagaccacatcttactgcccacctccatatgccccatcacccgggacaggccaggtgagaagtctgatggctgcgtgtccgccaagggcgaacttgtcatgcagtataagatcatccccaccccgggtacccaccccctgctggtcttggtgaaccccaagagtggagggagacaaggagaaagaattcttcggaaattccactatctgctcaaccccaaacaagttttcaacctggacaatggggggcctactccagggttgaactttttccgtgatactccagacttccgtgttttggcctgtggtggagatgggacagttggctggattttggattgcattgataaggccaactttgcaaagcatccaccagtggctgtcctgcctcttggaacaggaaatgaccttgcccgttgtctccgctggggaggaggttatgaagggggcagcttgacaaaaatcctgaaagacattgagcagagccccttggtgatgctggaccgctggcatctggaagtcatccccagagaggaagtggaaaacggggaccaggtcccatacagcatcatgaacaactatttctccattggtgtggacgcttccattgcacacagattccatgtgatgagagagaaacatcctgaaaaattcaacagcaggatgaagaacaagctgtggtactttgaatttggcacctcggagacttttgcagcgacctgcaagaaactccacgaccacattgagttggagtgtgatggggttggggtggacctgagcaacatcttcctggaaggcattgccattctcaacattcccagcatgtacggaggcaccaatctctggggagaaaacaagaagaaccgggctgtgatccgggaaagcaggaagggtgtcactgaccccaaagaactgaaattctgcgttcaagacctcagtgaccagctccttgaagtggtggggctagaaggagccatggagatggggcagatctacaccggcctgaagagtgcaggcaggaggctggcccagtgcgcctctgtcaccatcaggacaaacaagctgctgccaatgcaagtggatggagaaccctggatgcagccatgttgcacgattaaaattactcacaagaaccaagcgcccatgatgatggggcctccccagaagagcagcttcttctcgttgagaaggaagagccgttcaaaagactaaacagtgtgccaaacaccagctaaaccaagagagaaagcaagaaactataatgcacactcacacacaatttatgtgcacactcacacatgcacacacacacacacatacacactcttctctaaccagtggaagcaaagccacccttcgggaagaaaacgtcaccttgccatacattctgtttcaacagtgggtacacccctaacagagccagtgccaacaaaacattttgaatggacttagggcccatgaggttgtggctggcttaggcagcaacctccacattcccacaggccttgagcagaattttctgagactgaagggaaatccccctttctttctaccagccctgcaagtttcctcatggacgctcgcgaggagcaggctgcaggtttcctgcctatggtgagatcagatgtggccaagggaaggagctctggttccagagaatttgcacaaagttccctctgtacagagacaaaacggcctccggctctcagagcataatccttggcagggctcagcaggcgcacgttggtttcttggtcgtcctttgagtgacaacttctccgtgaacctgctgaagaggcagaaaggctgtggaaagctgtatttccattcttgggtttctgcgccgtcggtgggcacttgttattttccaggaaccttctcctggtgtctacatgtttgcttagaggcggctccaagagccccagagctgcctgcatagcacaccttagatgtggtatttattttcttagttctgtgaacacctgggagggagagcggagaaactgggatttatttttcaaattggtgtcataatattgtgtaaaaagggaaggaaaaaaaaaaccacccccagcttc

SEQ ID NO: 5 - DNA, 2470 bp, Homo sapiens
  gene (1)..(2470): The sequence of the cDNA coding for Dihydroxyacetone phosphate acyltransferase
gaattcggcacgagccgggatcctgtgtagcggctgcagagggtgccgccgccctaggcgaagtagggccgtcctgagcgaaagaaccgcccccagcaggagcaccaccacggcttagcaaagaatcccagaccccgcccgggaaggcagccgcaccatggagtcttccagttcatctaactcttatttctccgttggcccaaccagtcccagcgctgtcgtgctcctctactcgaaggagctcaaaaagtgggatgagtttgaagatattttagaagagaggaggcatgtcagtgacttgaaatttgcaatgaaatgctacacacctcttgtctataagggaattactccatgtaaaccaattgatattaaatgtagtgttctcaattctgaggagattcattatgtcattaaacagctttccaaggaatcccttcaatctgtggatgtcctccgagaggaagtgagtgagatcttagatgaaatgagtcacaaactgcgtcttggagccattcggttttgtgccttcaccctgagcaaagtatttaaacaaattttctcgaaggtgtgtgtaaatgaagaaggtattcagaaactacaaagagccatccaggagcatcctgttgttctgctgcctagtcatcgaagttacattgacttcctcatgttgtcttttcttctatacaattatgatttgcctgtgccagttatagcagcaggaatggacttcctgggaatgaaaatggttggtgagctgctacgaatgtcgggtgcctttttcatgcggcgtacctttggtggcaataaactctactgggctgtattctctgaatatgtaaaaactatgttacggaatggttatgctcctgttgaatttttcctcgaagggacaagaagccgctctgccaagacattgactcctaaatttggtcttctgaatattgtgatggagccattttttaaaagagaagtttttgatacctaccttgtcccaattagtatcagttatgataagatcttggaagaaactctttatgtgtatgagcttctaggggttcctaaaccaaaagagtctacaactgggttgctgaaagccagaaagattctctctgaaaattttggaagcatccatgtgtactttggagatcctgtgtcacttcgatctttggcagctgggaggatgagtcggagctcatataacttggttccaagatacattcctcagaaacagtctgaggacatgcatgcctttgtcactgaagttgcctacaaaatggagcttctgcaaattgaaaacatggttttgagcccctggaccctaatagttgctgttctgcttcagaaccggccatccatggactttgatgctctggtggaaaagactttatggctaaaaggcttaacccaggcatttggagggtttctcatttggcctgataataaacctgctgaagaagttgtcccggccagcattcttctgcattccaacattgccagccttgtcaaagaccaggtgattctgaaagtggactccggagactcggaagtggtcgatgggcttatgctccagcacatcactctcctcatgtgctcagcttataggaaccagctgctcaacatttttgtgcgcccatccttagtagcagtagcattgcagatgacaccagggttcaggaaagaggatgtctacagttgctttcgcttcctacgtgatgtttttgcagatgagttcatcttccttccaggaaacacactaaaggactttgaagaaggctgttacctgctttgtaaaagtgaagccatacaagtgactacgaaagacatcctagttacagagaaaggaaatactgtgttagaatttttagtaggactctttaaaccttttgtggaaagctatcagataatttgcaagtaccttttgagtgaagaagaggaccacttcagtgaggaacagtacttggctgcagtcagaaaattcacaagtcagcttctcgatcaaggtacctctcaatgttatgatgtattatcttctgatgtgcagaaaaacgccttagcagcctgtgtgaggctcggagtagtggagaagaagaagataaataataactgtatatttaatgtgaatgaacctgccacaaccaaattagaagaaatgcttggttgtaagacaccaataggaaaaccagccactgcaaaactttaataatcaacaaatagttatggaaaattcggtcacgtaattactctcatcgaaggactcattacaacaaacagggaagtaaaggaagagacacatcctctcatactccctgagactctgagaacagtggacgcagagggaagagatgatcattggaagcaatcagtttactcttccccaccacagtggttaaaaggcgtttgtatctgacactatgtgtgtgttttaaaataaacttttggaaacatgaaaaaaaaaaaaaaaaaactcgag

SEQ ID NO: 6 - DNA, 2757 bp, Homo sapiens
  gene (1)..(2757): The sequence of the cDNA coding for EDG-1
tctaaaggtcgggggcagcagcaagatgcgaagcgagccgtacagatcccgggctctccgaacgcaacttcgccctgcttgagcgaggctgcggtttccgaggccctctccagccaaggaaaagctacacaaaaagcctggatcactcatcgaaccacccctgaagccagtgaaggctctctcgcctcgccctctagcgttcgtctggagtagcgccaccccggcttcctggggacacagggttggcaccatggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaatgtggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcctagaactggaagctgtccacccaccggaagcgctctttacttggtcgctggccaccccagtgtttggaaaaaaatctctgggcttcgactgctgccagggaggagctgctgcaagccagagggaggaagggggagaatacgaacagcctggtggtgtcgggtgttggtgggtagagttagttcctgtgaacaatgcactgggaagggtggagatcaggtcccggcctggaatatatattctacccccctggagctttgattttgcactgagccaaaggtctagcattgtcaagctcctaaagggttcatttggcccctcctcaaagactaatgtccccatgtgaaagcgtctctttgtctggagctttgaggagatgttttccttcactttagtttcaaacccaagtgagtgtgtgcacttctgcttctttagggatgccctgtacatcccacaccccaccctcccttcccttcatacccctcctcaacgttcttttactttatactttaactacctgagagttatcagagctggggttgtggaatgatcgatcatctatagcaaataggctatgttgagtacgtaggctgtgggaagatgaagatggtttggaggtgtaaaacaatgtccttcgctgaggccaaagtttccatgtaagcgggatccgttttttggaatttggttgaagtcactttgatttctttaaaaaacatcttttcaatgaaatgtgttaccatttcatatccattgaagccgaaatctgcataaggaagcccactttatctaaatgatattagccaggatccttggtgtcctaggagaaacagacaagcaaaacaaagtgaaaaccgaatggattaacttttgcaaaccaagggagatttcttagcaaatgagtctaacaaatatgacatccgtctttcccacttttgttgatgtttatttcagaatcttgtgtgattcatttcaagcaacaacatgttgtattttgttgtgttaaaagtacttttcttgatttttgaatgtatttgtttcaggaagaagtcattttatggatttttctaacccgtgttaacttttctagaatccaccctcttgtgcccttaagcattactttaactggtagggaacgccagaacttttaagtccagctattcattagatagtaattgaagatatgtataaatattacaaagaataaaaatatattactgtctctttagtatggttttcagtgcaattaaaccgagagatgtcttgtttttttaaaaagaatagtatttaataggtttctgacttttgtggatcattttgcacatagctttatcaacttttaaacattaataaactgatttttttaaag

SEQ ID NO: 7 - DNA, 1217 bp, Homo sapiens
  gene (1)..(1217): The sequence of the cDNA coding for EDG-2
ctgacacctacagcatcaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgtcagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaggctcagaccgctcggcttcctccctcaaccacaccatcttggctggagttcacagcaatgatcactctgtggtttag

SEQ ID NO: 8 - DNA, 1137 bp, Homo sapiens
  gene (1)..(1137): The sequence of the cDNA coding for EDG-3
atggcaactgccctcccgccgcgtctccagccggtgcgggggaacgagaccctgcgggagcattaccagtacgtggggaagttggcgggcaggctgaaggaggcctccgagggcagcacgctcaccaccgtgctcttcttggtcatctgcagcttcatcgtcttggagaacctgatggttttgattgccatctggaaaaacaataaatttcacaaccgcatgtactttttcattggcaacctggctctctgcgacctgctggccggcatcgcttacaaggtcaacattctgatgtctggcaagaagacgttcagcctgtctcccacggtctggttcctcagggagggcagtatgttcgtggcccttggggcgtccacctgcagcttactggccatcgccatcgagcggcacttgacaatgatcaaaatgaggccttacgacgccaacaagaggcaccgcgtcttcctcctgatcgggatgtgctggctcattgccttcacgctgggcgccctgcccattctgggctggaactgcctgcacaatctccctgactgctctaccatcctgcccctctactccaagaagtacattgccttctgcatcagcatcttcacggccatcctggtgaccatcgtgatcctctacgcacgcatctacttcctggtgaagtccagcagccgtaaggtggccaaccacaacaactcggagcggtccatggcactgctgcggaccgtggtgattgtggtgagcgtgttcatcgcctgctggtccccactcttcatcctcttcctcattgatgtggcctgcagggtgcaggcgtgccccatcctcttcaaggctcagtggttcatcgtgttggctgtgctcaactccgccatgaacccggtcatctacacgctggccagcaaggagatgcggcgggccttcttccgtctggtctgcaactgcctggtcaggggacggggggcccgcgcctcacccatccagcctgcgctcgacccaagcagaagtaaatcaagcagcagcaacaatagcagccactctccgaaggtcaaggaagacctgccccacacagacccctcatcctgcatcatggacaagaacgcagcacttcagaatgggatcttctgcaactga

SEQ ID NO: 9 - DNA, 1056 bp, Homo sapiens
  gene (1)..(1056): The sequence of the cDNA coding for EDG-4
atggtcatcatgggccagtgctactacaacgagaccatcggcttcttctataacaacagtggcaaagagctcagctcccactggcggcccaaggatgtggtcgtggtggcactggggctgaccgtcagcgtgctggtgctgctgaccaatctgctggtcatagcagccatcgcctccaaccgccgcttccaccagcccatctactacctgctcggcaatctggccgcggctgacctcttcgcgggcgtggcctacctcttcctcatgttccacactggtccccgcacagcccgactttcacttgagggctggttcctgcggcagggcttgctggacacaagcctcactgcgtcggtggccacactgctggccatcgccgtggagcggcaccgcagtgtgatggccgtgcagctgcacagccgcctgccccgtggccgcgtggtcatgctcattgtgggcgtgtgggtggctgccctgggcctggggctgctgcctgcccactcctggcactgcctctgtgccctggaccgctgctcacgcatggcacccctgctcagccgctcctatttggccgtctgggctctgtcgagcctgcttgtcttcctgctcatggtggctgtgtacacccgcattttcttctacgtgcggcggcgagtgcagcgcatggcagagcatgtcagctgccacccccgctaccgagagaccacgctcagcctggtcaagactgttgtcatcatcctgggggcgttcgtggtctgctggacaccaggccaggtggtactgctcctggatggtttaggctgtgagtcctgcaatgtcctggctgtagaaaagtacttcctactgttggccgaggccaactcactggtcaatgctgctgtgtactcttgccgagatgctgagatgcgccgcaccttccgccgccttctctgctgcgcgtgcctccgccagtccacccgcgagtctgtccactatacatcctctgcccagggaggtgccagcactcgcatcatgcttcccgagaacggccacccactgatggactccaccctttag

SEQ ID NO: 10 - DNA, 1062 bp, Homo sapiens
  gene (1)..(1062): The sequence of the cDNA coding for EDG-5
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgcctccatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggcggccgggggtgggggtgcaaggacggaggcgggtcgggaccccgggccaccacctcctgccactccgcagctccagctccctggagaggggcatgcacatgcccacgtcacccacgtttctggagggcaacacggtggtctga

SEQ ID NO: 11 - DNA, 1566 bp, Homo sapiens
  gene (1)..(1566): The sequence of the cDNA coding for EDG-6
gagtcagcccccgggggaggccatgaacgccacggggaccccggtggcccccgagtcctgccaacagctggcggccggcgggcacagccggctcattgttctgcactacaaccactcgggccggctggccgggcgcggggggccggaggatggcggcctgggggccctgcgggggctgtcggtggccgccagctgcctggtggtgctggagaacttgctggtgctggcggccatcaccagccacatgcggtcgcgacgctgggtctactattgcctggtgaacatcacgctgagtgacctgctcacgggcgcggcctacctggccaacgtgctgctgtcgggggcccgcaccttccgtctggcgcccgcccagtggttcctacgggagggcctgctcttcaccgccctggccgcctccaccttcagcctgctcttcactgcaggggagcgctttgccaccatggtgcggccggtggccgagagcggggccaccaagaccagccgcgtctacggcttcatcggcctctgctggctgctggccgcgctgctggggatgctgcctttgctgggctggaactgcctgtgcgcctttgaccgctgctccagccttctgcccctctactccaagcgctacatcctcttctgcctggtgatcttcgccggcgtcctggccaccatcatgggcctctatggggccatcttccgcctggtgcaggccagcgggcagaaggccccacgcccagcggcccgccgcaaggcccgccgcctgctgaagacggtgctgatgatcctgctggccttcctggtgtgctggggcccactcttcgggctgctgctggccgacgtctttggctccaacctctgggcccaggagtacctgcggggcatggactggatcctggccctggccgtcctcaactcggcggtcaaccccatcatctactccttccgcagcagggaggtgtgcagagccgtgctcagcttcctctgctgcgggtgtctccggctgggcatgcgagggcccggggactgcctggcccgggccgtcgaggctcactccggagcttccaccaccgacagctctctgaggccaagggacagctttcgcggctcccgctcgctcagctttcggatgcgggagcccctgtccagcatctccagcgtgcggagcatctgaagttgcagtcttgcgtgtggatggtgcagccaccgggtgcgtgccaggcaggccctcctggggtacaggaagctgtgtgcacgcagcctcgcctgtatggggagcagggaacgggacaggcccccatggtcttcccggtggcctctcggggcttctgacgccaaatgggcttcccatggtcaccctggacaaggaggtaaccaccccacctccccgtaggagcagagagcaccctggtgtgggggcgagtggttccccacaaccccgcttctgtgtgattctggggaagtcccggcccctctctgggcctcagtagggctcccaggctgcaaggggtggactgtgggatgcatgccctggcaacattgaagttcgatcatggtaaaaaa

SEQ ID NO: 12 - DNA, 1148 bp, Homo sapiens
  gene (1)..(1148): The sequence of the cDNA coding for EDG-7
cttctttaaatttctttctaggatgttcacttcttctccacaatgaatgagtgtcactatgacaagcacatggactttttttataataggagcaacactgatactgtcgatgactggacaggaacaaagcttgtgattgttttgtgtgttgggacgtttttctgcctgtttatttttttttctaattctctggtcatcgcggcagtgatcaaaaacagaaaatttcatttccccttctactacctgttggctaatttagctgctgccgatttcttcgctggaattgcctatgtattcctgatgtttaacacaggcccagtttcaaaaactttgactgtcaaccgctggtttctccgtcaggggcttctggacagtagcttgactgcttccctcaccaacttgctggttatcgccgtggagaggcacatgtcaatcatgaggatgcgggtccatagcaacctgaccaaaaagagggtgacactgctcattttgcttgtctgggccatcgccatttttatgggggcggtccccacactgggctggaattgcctctgcaacatctctgcctgctcttccctggcccccatttacagcaggagttaccttgttttctggacagtgtccaacctcatggccttcctcatcatggttgtggtgtacctgcggatctacgtgtacgtcaagaggaaaaccaacgtcttgtctccgcatacaagtgggtccatcagccgccggaggacacccatgaagctaatgaagacggtgatgactgtcttaggggcgtttgtggtatgctggaccccgggcctggtggttctgctcctcgacggcctgaactgcaggcagtgtggcgtgcagcatgtgaaaaggtggttcctgctgctggcgctgctcaactccgtcgtgaaccccatcatctactcctacaaggacgaggacatgtatggcaccatgaagaagatgatctgctgcttctctcaggagaacccagagaggcgtccctctcgcatcccctccacagtcctcagcaggagtgacacaggcagccagtacatagaggatagtattagccaaggtgcagtctgcaataaaagcacttcctaaactctggatgcctctcggcccacccaggtgatgactgtcttagg

SEQ ID NO: 13 - DNA, 1606 bp, Homo sapiens
  gene (1)..(1606): The sequence of the cDNA coding for Glycerol-3-phosphate dehydrogenase
ttttttttttttttttttttttgggtggcggggggttgcaagtgggaagcctgctgttcagctgccggggctctccgcctccccccacctgtatgaggctgggtctggggaacctgtgctcagcattccaccccctggagcttgggcttggtcttccctgcgggtccctgcgctgacattcaggcggggagccaggaggcctggcgcgcctccagagcccgccgggggagccgggcgagggttctgggctctgacggcggggtcgcagggtcgcccgcctcctggacacgtctgtaggcctagggaagcctgccggccgggaggtacagagtaggagaagccagatcccagggcggacaacgagaagtcgtcaggctaagaaatggcatttcaaaaggcagtgaaagggacgattcttgttggaggaggtgctcttgcaactgttttaggactttctcagtttgctcattacagaaggaaacaaatgaacctggcctatgttaaagcagcagactgcatttcagaaccagttaacagggagcctccttccagagaagctcagctactgactttgcaaaatacatctgaatttgatatccttgttattggaggaggagcaacaggaagtggctgtgcgctagatgctgtcaccagaggactaaaaacagcccttgtagaaagagatgatttctcatcagggaccagcagcagaagcactaaattgatccatggtggtgtgagatatctgcagaaggccatcatgaagttggatattgagcagtataggatggtaaaagaagcccttcatgagcgtgccaacctgctagaaattgctccccatttatcagctccattgcctataatgcttccagtttacaagtggtggcagttaccttactactgggtaggaatcaagctgtatgatttggttgcaggaagcaattgcctaaaaagcagttatgtcctcagcaaatcaagagcccttgaacatttcccaatgctccagaaggacaaactggtaggagcaattgtctactatgacggacaacataacgatgcacggatgaaccttgccattgctctgactgctgccaggtatggggctgccacagccaattacatggaggtagtgagcttgctcaagaagacagacccccagacagggaaagtgcatgtgagcggcgcacggtgcaaggatgtcctcacagggcaggaatttgacgtgagagccaaatgtgttatcaatgccacgggacctttcacggactctgtgcgcaaaatggatgataaagacgcagcagctatctgccagccaagtgctggtgtccatattgtgatgcctggttattacagcccagagagcatgggacttcttgacccagcgaccagtgatgggcgagttattttcttcttaccctggcaaaagatgacgatcgctggcactactgatactccaactgatgttacacaccatccaattccttcagaagaagatatcaacttcattttgaatgaagtgcgtaattacctgagttgtgatgttgaagtgagaagaggggatgtcctggcagcatggagtggaatccgtcctcttgttacagacccc

SEQ ID NO: 14 - DNA, 2417 bp, Homo sapiens
  gene (1)..(2417): The sequence of the cDNA coding for Lyosphospholipase I
cttccttccgcttgcgctgtgagctgaggcggtgtatgtgcggcaataacatgtcaaccccgctgcccgccatcgtgcccgccgcccggaaggccaccgctgcggtgattttcctgcatggattgggagatactgggcacggatgggcagaagcctttgcaggtatcagaagttcacatatcaaatatatctgcccgcatgcgcctgttaggcctgttacattaaatatgaacgtggctatgccttcatggtttgatattattgggctttcaccagattcacaggaggatgaatctgggattaaacaggcagcagaaaatataaaagctttgattgatcaagaagtgaagaatggcattccttctaacagaattattttgggagggttttctcagggaggagctttatctttatatactgcccttaccacacagcagaaactggcaggtgtcactgcactcagttgctggcttccacttcgggcttcctttccacagggtcctatcggtggtgctaatagagatatttctattctccagtgccacggggattgtgaccctttggttcccctgatgtttggttctcttacggtggaaaaactaaaaacattggtgaatccagccaatgtgacctttaaaacctatgaaggtatgatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccgataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattttccctgatattatttctatttaatactcttttctctcaagaaaaaaaaaaaagtttgttttttctttattgtccttcatagcaggccaagtattgcctctctgcaatagacagctactgtcaatacatgctgtaatttgacattctgggtcacagatataaggtatttaaaatctatttatgctttatagagaaaccagacattaaaacttcatgcactacttatttcgaattactgtaccttatccaaatttacacctagctattaggatcttcaacccaggtaacaggaataattctgtggtttcatttttctgtaaacaactgaaagaataattagatcatattctagtatgttctgaaatatctttaagactgatcttaaaaactaacttctaagatgatttcatcttctcatagtatagagtttactttgtacacgttgaaaccaactactgtagaagatgaggaatctattgtaattttttgctttattttcatctgccagtggacttatttgaattttcactttagtcaaattattttttgtattagtttttgatgcagacataaaaatagcaatcattttaaattgtcaaaatttccagattactggtaaaaattatttgaaaacaaacttatgggtaataaaggctagtcagaaccctataccataaagtgtagttaccatacagattaatatgtagcaaaaatgtatgcttgatatttctcaactgtgttaatttttctgctgtattccagctgaccaaaacaatattaagaatgcatctttataaatgggtgctaattgataatggaaataatttagtaatggactatacaggatgttaataatgaagccatatgtttatgtctggatttaaaaattttaaacaatcatttactatgtcatttttctttaccttgaagaacataaactgttatttcacttctacaaatcagcaagatattatttatggcaagaaatattccattgaaatattgtgctgtaacatgggaaagtgtaaatgtttttcatggtttctatcaatgtgaaataaaatttaattctgaaaaa

SEQ ID NO: 15 - DNA, 1192 bp, Homo sapiens
  gene (1)..(1192): The sequence of the cDNA coding for Human Lysophospholipase Homolog
ccagcccgaaaggcagggtctgggtgcgggaagagggctcggagctgccttcctgctgccttggggccgcccagatgagggaacagcccgatttgcctggttctgattctccaggctgtcgtggttgtggaatgcaaacgccagcacataatggaaacaggacctgaagacccttccagcatgccagaggaaagttcccccaggcggaccccgcagagcattccctaccaggacctccctcacctggtcaatgcagacggacagtacctcttctgcaggtactggaaacccacaggcacacccaaggccctcatctttgtgtcccatggagccggagagcacagtggccgctatgaagagctggctcggatgctgatggggctggacctgctggtgttcgcccacgaccatgttggccacggacagagcgaaggggagaggatggtagtgtctgacttccacgttttcgtcagggatgtgttgcagcatgtggattccatgcagaaagactaccctgggcttcctgtcttccttctgggccactccatgggaggcgccatcgccatcctcacggccgcagagaggccgggccacttcgccggcatggtactcatttcgcctctggttcttgccaatcctgaatctgcaacaactttcaaggtccttgctgcgaaagtgctcaaccttgtgctgccaaacttgtccctcgggcccatcgactccagcgtgctctctcggaataagacagaggtcgacatttataactcagaccccctgatctgccgggcagggctgaaggtgtgcttcggcatccaactgctgaatgccgtctcacgggtggagcgcgccctccccaagctgactgtgcccttcctgctgctccagggctctgccgatcgcctatgtgacagcaaaggggcctacctgctcatggagttagccaagagccaggacaagactctcaagatttatgaaggtgcctaccatgttctccacaaggagcttcctgaagtcaccaactccgtcttccatgaaataaacatgtgggtctctcaaaggacagccacggcaggaactgcgtccccaccctgaatgcattggccggtgcccggctcatggtctgggggatgcaggcaggggaagggcagagatggcttctcagatatggcttgcaaaaaaaaaaaaaaaaaaa

SEQ ID NO: 16 - DNA, 2333 bp, Homo sapiens
  gene (1)..(2333): The sequence of the cDNA coding for N-acylsphingosine amidohydrolase
ggcacgaggctagagcgatgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccaatgctaaaggttatagtgaattctctgaagaatatgataaatacattcgtgccaagtggaaaagttatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgccatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcacacgtctggcctacagaatgcggcctctgagacatgaagacaccatctccatgtgaccgaacactgcagctgtctgaccttccaaagactaagactcgcggcaggttctctttgagtcaatagcttgtcttcgtccatctgttgacaaatgacagatctttttttttttccccctatcagttgatttttcttatttacagataacttctttaggggaagtaaaacagtcatctagaattcactgagttttgtttcactttgacatttggggatctggtgggcagtcgaaccatggtgaactccacctccgtggaataaatggagattcagcgtgggtgttgaatccagcacgtctgtgtgagtaacgggacagtaaacactccacattcttcagtttttcacttctacctacatatttgtatgtttttctgtataacagccttttccttctggttctaactgctgttaaaattaatatatcattatctttgctgttattgacagcgatattattttattacatatcattagagggatgagacagacattcacctgtatatttcttttaatgggcacaaaatgggcccttgcctctaaatagcactttttggggttcaagaagtaatcagtatgcaaagcaatcttttatacaataattgaagtgttccctttttcataattactctacttcccagtaaccctaaggaagttgctaacttaaaaaactgcatcccacgttctgttaatttagtaaataaacaagtcaaagacttgtggaaaataggaagtgaacccatattttaaattctcataagtagcattgatgtaataaacaggtttttagtttgttcttcagattgatagggagttttaaagaaattttagtagttactaaaattatgttactgtatttttcagaaatcaaactgcttatgaaaagtactaatagaacttgttaacctttctaaccttcacgattaactgtgaaatgtacgtcatttgtgcaagaccgtttgtccacttcattttgtataatcacagttgtgttcctgacactcaataaacagtcactggaaagagtgccagtcagcagtcatgcacgctgataaaaaaaaaaaaaaaaaa

SEQ ID NO: 17 - DNA, 1016 bp, Homo sapiens
  gene (1)..(1016): The sequence of the cDNA coding for Phospholipase A2
atggataccaatgttccgactggagacggggagcccgcgagacccgggtctccagggtctgcccaaggaagttgctcatgggagcagacccctagagcaggatttgaggccaggccaaagagaaccccagagatgaaaggcctcctcccactggcttggttcctggcttgtagtgtgcctgctgtgcaaggaggcttgctggacctaaaatcaatgatcgagaaggtgacagggaagaacgccctgacaaactacggcttctacggctgttactgcggctggggcggccgaggaacccccaaggatggcaccgattggtgctgttgggcgcatgaccactgctatgggcggctggaggagaagggctgcaacattcgcacacagtcctacaaatacagattcgcgtggggcgtggtcacctgcgagcccgggcccttctgccatgtgaacctctgtgcctgtgaccggaagctcgtctactgcctcaagagaaacctacggagctacaacccacagtaccaatactttcccaacatcctctgctcctaggcctccccagcgagctcctcccagaccaagacttttgttctgtttttctacaacacagagtactgactctgcctggttcctgagagaggctcctaagtcacagacctcagtctttctcgaagcttggcggacccccagggccacactgtaccctccagcgagtcccaggagagtgactctggtcataggacttggtagggtcccagggtccctaggcctccacttctgagggcagcccctctggtgccaagagctctcctccaactcagggttggctgtgtctcttttcttctctgaagacagcgtcctggctccagttggaacactttcctgagatgcacttacttctcagcttctgcgatcagattatcatcaccaccaccctccagagaattttacgcaagaagagccaaattgactctctaaatctggtgtatgggtattaaataaaattcattctcaaggct

SEQ ID NO: 18 - DNA, 3609 bp, Homo sapiens
  gene (1)..(3609): The sequence of the cDNA coding for Phospholipase D1 (phosphatidylcholine specific)
ggcacgaggagccctgagagtccgccgccaacgcgcaggtgctagcggccccttcgccctgcagcccctttgcttttactctgtccaaagttaacatgtcactgaaaaacgagccacgggtaaatacctctgcactgcagaaaattgctgctgacatgagtaatatcatagaaaatctggacacgcgggaactccactttgagggagaggaggtagactacgacgtgtctcccagcgatcccaagatacaagaagtgtatatccctttctctgctatttataacactcaaggatttaaggagcctaatatacagacgtatctctccggctgtccaataaaagcacaagttctggaagtggaacgcttcacatctacaacaagggtaccaagtattaatctttacactattgaattaacacatggggaatttaaatggcaagttaagaggaaattcaagcattttcaagaatttcacagagagctgctcaagtacaaagcctttatccgcatccccattcccactagaagacacacgtttaggaggcaaaacgtcagagaggagcctcgagagatgcccagtttgccccgttcatctgaaaacatgataagagaagaacaattccttggtagaagaaaacaactggaagattacttgacaaagatactaaaaatgcccatgtatagaaactatcatgccacaacagagtttcttgatataagccagctgtctttcatccatgatttgggaccaaagggcatagaaggtatgataatgaaaagatctggaggacacagaataccaggcttgaattgctgtggtcagggaagagcctgctacagatggtcaaaaagatggttaatagtgaaagattcctttttattgtatatgaaaccagacagcggtgccattgccttcgtcctgctggtagacaaagaattcaaaattaaggtggggaagaaggagacagaaacgaaatatggaatccgaattgataatctttcaaggacacttattttaaaatgcaacagctatagacatgctcggtggtggggaggggctatagaagaattcatccagaaacatggcaccaactttctcaaagatcatcgatttgggtcatatgctgctatccaagagaatgctttagctaaatggtatgttaatgccaaaggatattttgaagatgtggcaaatgcaatggaagaggcaaatgaagagatttttatcacagactggtggctgagtccagaaatcttcctgaaacgcccagtggttgagggaaatcgttggaggttggactgcattcttaaacgaaaagcacaacaaggagtgaggatcttcataatgctctacaaagaggtggaactcgctcttggcatcaatagtgaatacaccaagaggactttgatgcgtctacatcccaacataaaggtgatgagacacccggatcatgtgtcatccaccgtctatttgtgggctcaccatgagaagcttgtcatcattgaccaatcggtggcctttgtgggagggattgacctggcctatggaaggtgggacgacaatgagcacagactcacagacgtgggcagtgtgaagcgggtcacttcaggaccgtctctgggttccctcccacctgccgcaatggagtctatggaatccttaagactcaaagataaaaatgagcctgttcaaaacctacccatccagaagagtattgatgatgtggattcaaaactgaaaggaataggaaagccaagaaagttctccaaatttagtctctacaagcagctccacaggcaccacctgcacgacgcagatagcatcagcagcattgacagcacctccagttattttaatcactatagaagtcatcacaatttaatccatggtttaaaaccccacttcaaactctttcacccgtccagtgagtctgagcaaggactcactagacctcatgctgataccgggtccatccgtagtttacagacaggtgtgggagagctgcatggggaaaccagattctggcatggaaaggactactgcaatttcgtcttcaaagactgggttcaacttgataaaccttttgctgatttcattgacaggtactccacgccccggatgccctggcatgacattgcctctgcagtccacgggaaggcggctcgtgatgtggcacgtcacttcatccagcgctggaacttcacaaaaattatgaaatcaaaatatcggtccctttcttatccttttctgcttccaaagtctcaaacaacagcccatgagttgagatatcaagtgcctgggtctgtccatgctaacgtacagttgctccgctctgctgctgattggtctgctggtataaagtaccatgaagagtccatccacgccgcttacgtccatgtgatagagaacagcaggcactatatctatatcgaaaaccagtttttcataagctgtgctgatgacaaagttgtgttcaacaagataggcgatgccattgcccagaggatcctgaaagctcacagggaaaaccagaaataccgggtatatgtcgtgataccacttctgccagggttcgaaggagacatttcaaccggcggaggaaatgctctacaggcaatcatgcacttcaactacagaaccatgtgcagaggagaaaattccatccttggacagttaaaagcagagcttggtaatcagtggataaattacatatcattctgtggtcttagaacacatgcagagctcgaaggaaacctagtaactgagcttatctatgtccacagcaagttgttaattgctgatgataacactgttattattggctctgccaacataaatgaccgcagcatgctgggaaagcgtgacagtgaaatggctgtcattgtgcaagatacagagactgttccttcagtaatggatggaaaagagtaccaagctggccggtttgcccgaggacttcggctacagtgctttagggttgtccttggctatcttgatgacccaagtgaggacattcaggatccagtgagtgacaaattcttcaaggaggtgtgggtttcaacagcagctcgaaatgctacaatttatgacaaggttttccggtgccttcccaatgatgaagtacacaatttaattcagctgagagactttataaacaagcccgtattagctaaggaagatcccattcgagctgaggaggaactgaagaagatccgtggatttttggtgcaattccccttttatttcttgtctgaagaaagcctactgccttctgttgggaccaaagaggccatagtgcccatggaggtttggacttaagagatattcattggcagctcaaagacttccaccctggagaccacactgcacacagtgacttcctggggatgtcatagccaaagccaggcctgacgcattctcgtatccaacccaaggaccttttggaatgactggggagggctgcagtcacattgatgtaaggactgtaaacatcagcaagactttataattccttctgcctaacttgtaaaaagggggctgcattcttgttggtagcatgtactctgttgagtaaaacacatattcaaattccgctcgtgccgaattc

SEQ ID NO: 19 - DNA, 2893 bp, Homo sapiens
  gene (1)..(2893): The sequence of the cDNA coding for Phospholipase D1 glycosylphosphatidylinositol specific
cgtcattagaggagccggtggggaatgagagcatgtctgctttcaggttgtggcccggcctgctgatgatcgtgatggcttctctctgccatagaggttcatcgtgtggcctttcaacgcacatagaaatcggacacagagctctggagtttcttcatcttcacaatgggcatgttaactacaaagagctgttactagaacaccaggatgcatatcaggctggaaccgtgtttcctgattgtttttaccctagcctctgcaaaggaggaaaattccatgatgtgtctgagagcactcactggactccgtttcttaacgcaagcgttcattatatccgagagaactatccccttccctgggagaaggacacagagaaactggtagctttcttgtttggaattacttctcatatggtagcagatgtcagctggcatagtctgggcattgaacaaggattccttaggaccatgggagctattgattttcacggctcctattctgaggctcattcagctggtgattttggaggagatgtgttgagccagtttgaatttaattttaattaccttgcacgacgctggtatgtgccagtcaaagatctgctgggaatttatgagaaactctatggtcgagaagtcatcactgaaaatgtaattgttgattgttcacatatccagttcttagaaatgtatggtgagatgctagctgtttccaagttatatccctcttactctacaaagtccccgtttttggtggaacaattccaagagtattttcttggaggactggatgatatggcgttttggtccactaatatttaccatctaacgagcttcatgttggagaatgggaccagtgactgcagcctacctgagaaccctctgttcattgcatgtggtggccagcaaaaccacacccagggctcgaaaatgcagaaaaatgattttcacagaaatttgacttcatccctaactgaaaacattgacaggaatataaactataccgaaagaggagtgttcttcagtgtaaattcctggaccccggattccatgtcctttatctacaaggctttggaaaggaacgtaaggacaatgttcataggtggctctcagttgtcacagaagcacatctctagccccttagcatcttacttcttgtcatttccttatgcaaggcttggctgggcaatgacctcagctgacctcaaccaggatgggtacggcgacctcgtggtgggcgcaccaggctacagccgccctggccgcatccacatcgggcgcgtgtacctcatctacggcaatgaactgggtctgccgcccgttgacctggacctggacaaggaggcccacgggatccttgaaggtttccagccctcaggtcggtttggctcggccttggctatgttggactttaacatggatggcgtgcctgacctggccgtgggagctccctcggtgggctctgagcagctcacctacaaaggtgctgtgtatgtctactttggttccaaacaaggaagaatgtcttcttcccctaacatcaccatctcttgccaggacatctactgtaacttgggctggactctcttggctgcagatgtgaatggagacagtgagcccgatctggtcattggctccccttttgcaccaggtggagggaagcagaagggaattgtggctgcgttttattctggccccagcctgagcaacaaagagaaactgaacgtggaggcggccaactggacggtgagaggcgaggaagactttgcctggtttggatactcccttcacggtgtcactgtggacaacagaaccttgctgctggttgggagcccgacctggaagaatgccagcaggctgggccgtttgttacacatccgagatgagaaaaagagccttgggagggtgtatggctacttcccaccaaacagccaaagctggtttaccattgttggagacaaggcaatggggaaactgggtacttccctgtccagtggccacgtgctgatgaatggaactctgacccaggtgctgctggtgggagccccgacacgtgatgatgtgtctaagatggcattcctgaccatgaccctgcaccaaggcggagccactcggatgtacgcgctcacatccgacctgcagccaccgctgctcagcaccttcagcggagaccgccgcttctctcgatttggtggcgttctgcacttgagtgacctggatgatgatggcgtagatgaaatcatcgtggcagcccccctgaggatagcagatgtaacctctgggctgattgggggagaagatggccgagtttatgtatataatggcaaagagaccacccttggtgacatgactggcaaatgcaaatcgtggatgactccatgtccagaagaaaaggcccaatatgtattgatttctcctgaagccagctcaaggtttgggagctccctgatcaccgtgaggtccaaggcaaagaatcaagtcgtcattgccgctggaaggagctctttgggagcccgactctccggggcacttcacgtctatagctttggctcagattgaagatttcactgcgtttccccactctgcccacctctctcatgctgaatcacatccatggtgagcattttgatggacaaaatggcacatccagtggagctgtggcagatcctaatagatgtggggctcctgggagtagagacacacaccaacagccaccctttctggaaatctgatatagtatatatatgactgcaccaggagtatgtgaaatatcagacacactctgctcattcatgtctccttccacagtttatttcctcgcttcctttgcatctaaacctttcttctttccgaactttttgcctatagtcagacctgctgtaccacctatttcc

SEQ ID NO: 20 - DNA, 1362 bp, Homo sapiens
  gene (1)..(1362): The sequence of the cDNA coding for Phosphatidic Acid Phosphatase type 2B
ggcgcagctctgcaaaagtttctgctcgggatctggctctcttccccttggactttagaacgatttagggttgacagaggaaagcagaggcgcgcaggaggagcagaaaacaccaccttctgcagttggaggcaggcagccccggctgcactctagccgccgcgcccggagccggggccgacccgccactatccgcagcagcctcggccaggaggcgacccgggcgcctgggtgtgtggctgctgttgcgggacgtcttcgcggggcgggaggctcgcgccgcagccagcgccatgcaaaactacaagtacgacaaagcgatcgtcccggagagcaagaacggcggcagcccggcgctcaacaacaacccgaggaggagcggcagcaagcgggtgctgctcatctgcctcgacctcttctgcctcttcatggcgggcctccccttcctcatcatcgagacaagcaccatcaagccttaccaccgagggttttactgcaatgatgagagcatcaagtacccactgaaaactggtgagacaataaatgacgctgtgctctgtgccgtggggatcgtcattgccatcctcgcgatcatcacgggggaattctaccggatctattacctgaagaagtcgcggtcgacgattcagaacccctacgtggcagcactctataagcaagtgggctgcttcctctttggctgtgccatcagccagtctttcacagacattgccaaagtgtccatagggcgcctgcgtcctcacttcttgagtgtctgcaaccctgatttcagccagatcaactgctctgaaggctacattcagaactacagatgcagaggtgatgacagcaaagtccaggaagccaggaagtccttcttctctggccatgcctccttctccatgtacactatgctgtatttggtgctatacctgcaggcccgcttcacttggcgaggagcccgcctgctccggcccctcctgcagttcaccttgatcatgatggccttctacacgggactgtctcgcgtatcagaccacaagcaccatcccagtgatgttctggcaggatttgctcaaggagccctggtggcctgctgcatagttttcttcgtgtctgacctcttcaagactaagacgacgctctccctgcctgcccctgctatccggaaggaaatcctttcacctgtggacattattgacaggaacaatcaccacaacatgatgtaggtgccacccacctcctgagctgtttttgtaaaatgactgctgacagcaagttcttgctgctctccaatctcatcagacagtagaatgtagggaaaaacttttgcccgactgatttttaaaaaaaaaaaaaaaa

SEQ ID NO: 21 - DNA, 1043 bp, Homo sapiens
  gene (1)..(1043): The sequence of the cDNA coding for Phosphatidic Acid Phosphatase type 2a
cccggcccgggctcgagaatcaagggcctcggccgccgtcccgcagctcagtccatcgcccttgccgggcagcccgggcagagaccatgtttgacaagacgcggctgccgtacgtggccctcgatgtgctctgcgtgttgctggctggattgccttttgcaatttttacttcaaggcatattacttcaaggcatacccccttccaacgaggagtattctgtaatgatgagtccatcaagtacccttacaaagaagacaccataccttatgcgttattaggtggaataatcattccattcagtattatcgttattattcttggagaaaccctgtctgtttactgtaaccttttgcactcaaattcctttatcaggaataactacatagccactatttacaaagccattggaacctttttatttggtgcagctgctagtcagtccctgactgacattgccaagtattcaataggcagactgcggcctcacttcttggatgtttgtgatccagattggtcaaaaatcaactgcagcgatggttacattgaatactacatatgtcgagggaatgcagaaagagttaaggaaggcaggttgtccttctattcaggccactcttcgttttccatgtactgcatgctgtttgtggcactttatcttcaagccaggatgaagggagactgggcaagactcttacgccccacactgcaatttggtcttgttgccgtatccatttatgtgggcctttctcgagtttctgattataaacaccactggagcgatgtgttgactggactcattcagggagctctggttgcaatattagttgctgtatatgtatcggatttcttcaaagaaagaacttcttttaaagaaagaaaagaggaggactctcatacaactctgcatgaaacaccaacaactgggaatcactatccgagcaatcaccagccttgaaaggcagcagggtgcccaggtgaagctggcctgttttctaaaggaaaatgattgccacaaggcaagaggatgcatctttcttcctgg

SEQ ID NO: 22 - DNA, 5397 bp, Homo sapiens
  gene (1)..(5397): The sequence of the cDNA coding for Phosphatidylinositol-3-Kinase (class 2, gamma polypeptide)
gaattcggcacgagcacttccttctcggctagattatctgaaactgttgtcggttcttgagatgatactaccaccgaatgtctgtgtttcattgtctagtccaacctgtattgtggatatctacaacgttccggcaatagttttgcaggtgcatcacatttttgtttttgttttgggaggaaaagggagggcacggcagccaggcttcatattcctacaagtgcatgcttcaagattactgtacttacagtgtttccaacatcttctcataaaaggggaaagcttcatagcctcaaccatgaaggaaaccagtcgcatagggcatggagctggagaactataaacagcccgtggtgctgagagaggacaactgccgaaggcgccggaggatgaagccgcgcagtgctgccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggacgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcggcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattctcggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaacctttgtctcattccttaaatgatgcttccaaacatctccttagtgtctgcaggtgttagtggtgtgctaaaagcaaggaaagcgagttagtcttttcagtgtcttttgcaattcaattcttttgtcatgtataactgagacacacaaacacagcaggagaaatctaaaccgttgtgccttgaccttcctctgctggtcttgttccagggttatgaatatgaaaaaatagagatgagactttttgtgtcaactctgtccacaagagtgagttatctagtatgattagtatagctttctccagcatggcagcaggaagtaactacagggcctcttttatgcctgacatttcttcccttcctttttccctgcctccctttttcatcaattgcaatgctcccacaactctttacagacttgtgaaatcttcaagaacacctttactctataactcaaaaattagttgaaaaataattacttctcaaggattattagaatcttaggtacttatttgtaaagatgtttagtgacttttttttcaagtatctataaaggaggcagattctagaaaatatgaattagtttccaaatgccttaattttaaactttggcctgaacagttttttctttttcttaatggaagaagatatttaatatcttaaaaatattccaagttaggaagaacactacttgccttatccatttcccatttaaaggacttttaaactttgacacagtccttcagatttcctgaaaatccttgaaatatcttactttaaaaatattttcatctctgaaatatctcgttatttattggaggtattgtttaaccttagatagaccattaaattatttataaaatattttgtaattactgtagctaatacattacatagaaaaaactatgttaacagtgtctctgtttaagtataatcagatataaatatataacttaattttttaattttaaaaaatagatacctgtttgactttgaggtagtccaggcctttttctttttttttttttttaatgtgtgcaaaagcccaaaggttcctaagcctggctgcaaagaagaatcaacagggacactttttaaaaacactcttatcagcctggggcaacacagtgagactccatctcttaaaaaaaaaattagctgggtatagtggtatgtgcctgtagtcccaggtactcaggaggctgaggcaggaggattgcctgagcccaggaggtggaaactgcagagagtcatgatcatgtccttacactccagcctggataacagagcgagaccctgtctcaaaaaaaaaaaaaaaaaaaaaaaaaaaactcgag

SEQ ID NO: 23 - DNA, 3424 bp, Homo sapiens
  gene (1)..(3424): The sequence of the cDNA coding for Phosphatidylinositol-3-kinase (catalytic, alpha polypeptide)
aggatcagaacaatgcctccaagaccatcatcaggtgaactgtggggcatccacttgatgcccccaagaatcctagtggaatgtttactaccaaatggaatgatagtgactttagaatgcctccgtgaggctacattagtaactataaagcatgaactatttaaagaagcaagaaaataccctctccatcaacttcttcaagatgaatcttcttacattttcgtaagtgttacccaagaagcagaaagggaagaattttttgatgaaacaagacgactttgtgatcttcggctttttcaaccatttttaaaagtaattgaaccagtaggcaaccgtgaagaaaagatcctcaatcgagaaattggttttgctatcggcatgccagtgtgcgaatttgatatggttaaagatcctgaagtacaggacttccgaagaaatattcttaatgtttgtaaagaagctgtggatcttagggatcttaattcacctcatagtagagcaatgtatgtctatccgccacatgtagaatcttcaccagagctgccaaagcacatatataataaattggatagaggccaaataatagtggtgatttgggtaatagtttctccaaataatgacaagcagaagtatactctgaaaatcaaccatgactgtgtgccagaacaagtaattgctgaagcaatcaggaaaaaaactagaagtatgttgctatcatctgaacaattaaaactctgtgttttagaatatcagggcaagtacattttaaaagtgtgtggatgtgatgaatacttcctagaaaaatatcctctgagtcagtataagtatataagaagctgtataatgcttgggaggatgcccaatttgaagatgatggctaaagaaagcctttattctcaactgccaatggactgttttacaatgccatcttattccagacgcatttccacagctacaccatatatgaatggagaaacatctacaaaatccctttgggttataaatagagcactcagaataaaaattctttgtgcaacctacgtgaatctaaatattcgagacattgacaagatttatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaggaaggatgaaacacaaaaggtacagatgaagtttttagttgagcaaatgaggcgaccagatttcatggatgccctacagggcttgctgtctcctctaaaccctgctcatcaactaggaaacctcaggcttaaagagtgtcgaattatgtcttctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaagataactgagaaaatgaaagctcactctggattccacactgcactgttaataactctcagcaggcaaagaccgattgcataggaattgcacaatccatgaacagcattagatttacagcaagaacagaaataaaatactatataatttaaataatgtaaacgcaaacagggtttgatagcacttaaactagttcatttcaaaa

SEQ ID NO: 24 - DNA, 1201 bp, Homo sapiens
  gene (1)..(1201): The sequence of the cDNA coding for Prostate Differentiation Factor PLAB
agtcccagctcagagccgcaacctgcacagccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcacaccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgacctctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggaggcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 25 - DNA, 1269 bp, Homo sapiens
  gene (1)..(1269): The sequence of the cDNA coding for Phosphatidic Acid Phosphatase type 2c
gcgacgggacgcgctgggaccggcgtcgggggtcgcggggaccatgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctgctgtgagtccagctgatgcccacccaggtggtccctccagcctggttaggcactgagggttctggacgggctccaggaaccctgggctgatgggagcagtgagcggttccgctgccccctgccctgcactggaccaggagtctggagatgcctgggtagccctcagcatttggaggggaacctgttcccgtcggtccccaaatatccccttctttttatggggttaaggaagggaccgagagatcagatagttgctgttttgtaaaatgtaatgtatatgtggtttttagtaaaatagggcacctgtttcacaaaaaaaaaaaaaaaaaaa

SEQ ID NO: 26 - DNA, 1286 bp, Homo sapiens
  gene (1)..(1286): The sequence of the cDNA coding for Phosphocholine cytidyltransferase
cgaccggaccgggctcgggggagcgtgagttgcagttaaaagaagatggatgcacagtgttcagccaaggtcaatgcaaggaagaggagaaaagaggcgcccggacccaacggggcaacagaagaagatggggttccttccaaagtgcagcgctgtgcagtgggcttacggcaaccagctcctttttctgatgaaattgaagttgactttagtaagccctatgtcagggtaactatggaagaagccagcagaggaactccttgtgagcgacctgtgagagtttatgccgatggaatatttgacttatttcactctggtcacgcccgagctctgatgcaagcgaagaaccttttccctaatacgtacctcattgtgggagtttgcagtgatgagctcacacacaacttcaaaggcttcacggtgatgaacgagaatgagcgctatgacgcagtccagcactgccgctacgtggatgaggtggtgaggaatgcgccctggacgctgacacccgagttcctggccgaacaccggattgattttgtagcccatgatgatattccttattcatctgctggcagtgatgatgtttataagcacatcaaggaggcaggcatgtttgctccaacacagaggacagaaggtatctccacatcagacatcatcacccgaattgtgcgggattatgatgtgtatgcgaggcggaacctgcagaggggctacacagcaaaggagctcaatgtcagctttatcaacgagaagaaataccacttgcaggagagggttgacaaagtaaaggagaaagtgaaagatgtggaggaaaagtcaaaagaatttgttcagaaggtggaggaaaaaagcattgacctcattcagaagtgggaggagaagtcccgagaattcattggaagttttctggaaatgtttggtccggaaggagcactgaaacatatgctgaaagaggggaagggccggatgctgcaggccatcagcccgaagcagagccccagcagcagccctactcgcgagcgctccccctccccctctttccgatggcccttctccggcaagacttccccaccttgctccccagcaaatctctccaggcacaaggctgcagcctatgatatcagtgaggatgaagaagactaatgtttcctccctcctttcctgtcctccctttctgtcccattaccttcagaagctctctgttgaattccgaattgtgaccccaacactaaacctaaggacagctacaaaggaaagacaactggggaaagaagacctag

SEQ ID NO: 27 - DNA, 1856 bp, Homo sapiens
  gene (1)..(1856): The sequence of the cDNA coding for Phosphate cytidylyltransferase 2 (ethanolamine specific)
attgcgggcggcggcgttcggagtcgccgggagctgccaggctgtccgcgccgccgctgcggggccatgatccggaacgggcgcggggctgcaggcggcgcagagcagccgggcccggggggcaggcgcgccgtgagggtgtggtgcgatggctgctatgacatggtgcattacggccactccaaccagctgcgccaggcacgggccatgggtgactacctcatcgtaggcgtgcacaccgatgaggagatcgccaagcacaaggggcccccggtgttcactcaggaggagagatacaagatggtgcaggccatcaaatgggtggacgaggtggtgccagcggctccctacgtcactacactagagaccctggacaaatacaactgtgacttctgtgttcacggcaatgacatcaccctgactgtagatggccgggacacctatgaggaagtaaagcaggctgggaggtacagagaatgcaagcgcacgcaaggggtgtccaccacagacctcgtgggccgcatgctgctggtaaccaaagcccatcacagcagccaggagatgtcctctgagtaccgggagtatgcagacagttttggcaagtgccctggtgggcggaacccctggaccggggtatcccagttcctgcagacatctcagaagatcatccagtttgcttctgggaaggagccccagccaggggagacagtcatctatgtggctggtgccttcgacctgttccacatcgggcatgtggacttcctggagaaggtgcacaggctggcagagaggccctacatcatcgcgggcttacactttgaccaggaggtcaatcactacaaggggaagaactaccccatcatgaatctgcatgaacggactctgagcgtgctggcctgccggtacgtgtcagaagtggtgattggagccccgtacgcggtcacagcagagctcctaagtcacttcaaggtggacctggtgtgtcacggcaagacagaaattatccctgacagggatggctccgacccataccaggagcccaagagaaggggcatcttccgtcagattgacagtggcagcaacctcaccacagacctcatcgtccagcggatcatcaccaacaggttggagtatgaggcgcgaaaccagaagaaggaagccaaggagctggccttcctggaggctgccaggcagcaggcggcacagcccctgggggagcgcgatggtgacttctaacctggcagaggccctggccggccctccccctgctctgcttctgcgccttctgcgtttggacataggactctgcagggccgccctctctaactggcctggctctggaagggctggtgaggactctgcctccttgcctgcctacaaggtgcctggtttgcagcaggctctccgctctttccagcaaagctgctcagagagggtgtccagcacagtggagaggccggaagtgagacgggcagacggcacctgcagcctgaaacgcaccgctcctgcgtgcgcccccacctggtccccggatgcccccaccacctggacagaggccacactgactgcccacccagctgtggcgggaggtgcagagcagggggctttagggagcagtgactgcggtcacccctttagttctctgggtgtagaccacaccacctcccactgggcaccccccaacacggtgtcctgccacccagcgcctggctccaggaaaacacgcttgccttccttcccggcagcttcgccactctccttatggactctgttctgtttgtacatggctgacggaaatctctttggtacaaccgaataaagcctggtggcagtgctgcgcggggctcccagccaat

SEQ ID NO: 28 - DNA, 3160 bp, Homo sapiens
  gene (1)..(3160): The sequence of the cDNA coding for Phosphatase and Tenson Homolog (PTEN)
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 29 - DNA, 1707 bp, Homo sapiens
  gene (1)..(1707): The sequence of the cDNA coding for Sphingosine-1-phosphate lyase 1
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactcatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgagggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactga

SEQ ID NO: 30 - DNA, 1879 bp, Homo sapiens
  gene (1)..(1879): The sequence of the cDNA coding for Sphingomyelin phosphodiesterase 1
cctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcatacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccaggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagggccccagggcccacatttgggaaagttcttgatgtaggaaagggtgaaaaagcccaaatgctgctgtggttcaaccaggcaagatcatccggtgaaagaaccagtccctgggccccaaggatgccggggaaacaggaccttctcctttcctggagctggtttagctggatatgggagggggtttggctgcctgtgcccaggagctagactgccttgaggctgctgtcctttcacagccatggagtagaggcctaagttgacactgccctgggcagacaagacaggagctgtcgccccaggcctgtgctgcccagccaggaaccctgtactgctgctgcgacctgatgctgccagtctgttaaaataaag

SEQ ID NO: 31 - DNA, 3553 bp, Homo sapiens
  gene (1)..(3553): The sequence of the cDNA coding for Phospholipase C beta 3 (phosphatidylinositol specific)
gaagcgggtggagactgcgctggaatcctgtggcctcaaattcaaccggagtgagtccatccggcctgatgagttttccttggaaatctttgagcggttcctgaacaagctgtgtctgcggccggacattgacaagatcctgctggagataggcgccaagggcaagccatacctgacgctggagcagctcatggacttcatcaaccagaagcaacgcgacccgagactcaacgaagtgctgtacccgcccctgcggccctcccaggcccggctgctcatcgaaaagtatgagcccaaccagcagtttctggagcgagaccagatgtccatggagggctttagccgctacctgggaggcgaggagaatggcatcctgcccctggaagccctggatctgagcacggacatgacccagccactgagtgcctacttcatcaactcctcgcataacacctatctcactgcggggcagctggctgggacctcgtcggtggagatgtaccgccaggcactactatggggctgccgctgcgtggagctggacgtgtggaagggacggccgcctgaggaggaacccttcattacccacggcttcaccatgaccacagaggtgcctctgcgcgacgtgctggaggccattgccgagactgccttcaagacctcgccctaccccgtcatcctctccttcgagaaccatgtggactcggcaaagcaacaggcaaagatggctgagtactgccgctccatctttggagacgcgctactcatcgagcctctggacaagtacccgctggccccaggcgttcccctgcccagcccccaggacctgatgggccgtatcctggtgaagaacaagaagcggcaccgacccagcgcaggtggcccagacagcgccgggcgcaagcggcccctggagcagagcaattctgccctgagcgagagctccgcggccaccgagccctcctccccgcagctggggtctcccagctctgacagctgcccaggcctgagcaatggggaggaggtagggcttgagaagcccagcctggagcctcagaagtctctgggtgacgagggcctgaaccgaggcccctatgttcttggacctgctgaccgtgaggatgaggaggaagatgaggaagaggaggaacagacagaccccaaaaagccaactacagatgagggcacagccagcagcgaggtgaatgccactgaggagatgtccacgcttgtcaactacatcgaacctgtcaagttcaagtcctttgaggctgctcgaaagaggaacaaatgcttcgagatgtcgtcctttgtggagaccaaggccatggagcaactgaccaagagccccatggagtttgtggaatacaacaagcagcagctcagccgcatctaccccaagggcacccgcgtggactcctccaactacatgccccagctcttctggaacgtagggtgccagcttgttgcgctcaacttccagaccctcgatgtggcgatgcagctcaacgcgggcgtttttgagtacaacgggcgcagcgggtacctgctcaagccggagttcatgcggcggccggacaagtccttcgaccccttcactgaggtcatcgtggatggcatcgtggccaatgccttgcgggtcaaggtgatctcagggcagttcctgtccgacaggaaggtgggcatctacgtggaggtggacatgtttggcctccctgttgatacgcggcgcaagtaccgcacccggacctctcaggggaactcgttcaaccccgtgtgggacgaagagcccttcgacttccccaaggtggtgctgcccacgctggcttcacttcgcattgcagcctttgaggaggggggtaaattcgtagggcaccggatcctgcctgtctctgccatccgctccggataccactacgtctgcctgcggaacgaggccaaccaaccgctgtgcctgccggccctgctcatctacaccgaagccttggactacattcctgacgaccaccaggactatgcggaggccctgatcaaccccattaagcacgtcagcctgatggaccagagggcccggcagctggccgccctcattggggagagtgaggctcaggctggccaagagacgtgccaggacacccagtctcagcagctggggtctcagccgtcctcaaaccccacccccagcccactggatgcctccccccgccggccccctggccccaccacctcccctgccagcacctccctcagcagcccagggcagcgtgatgatctcatcgccagcatcctctcagaggtggcccccaccccgctggatgagctccgaggtcacaaggctctggtcaagctccggagccggcaagagcgagacctgcgggagctgcgcaagaagcatcagcggaaggcagtcaccctcacccgccgcctgctggatggcctggctcaggcacaggctgagggcaggtgccggctgcggccaggtgccctaggtggggccgctgatgtggaggacacgaaggagggggaggacgaggcaaagcggtatcaggagttccagaacagacaggtgcagagcctgctggagctgcgggaggcccaggtggacgcagaggcccagcggaggctggaacacctgagacaggctctgcagcggctcagggaggtcgtccttgatgcaaacacaactcagttcaagaggctgaaagagatgaacgagagggagaagaaggagctgcagaagatcctggacagaaagcgccataacagcatctcggaggccaagatgagggacaagcataagaaggaggcggaactgacggagattaaccgtcggcacatcactgagtcagtcaactccatccgtcggctggaggaggcccagaagcagcggcatgaccgtcttgtggctgggcagcagcaggtcctgcaacagctggcagaagaggagcccaagctgctggcccagctggcccaggagtgtcaggagcagcgggcgaggctcccccaggagatccgccggagcctgctgggcgagatgccggaggggctgggggacgggcctctggtggcctgtgccagcaacggtcacgcacccgggagcagcgggcacctgtcgggcgctgactcggagagccaggaggagaacacgcagctctgaactggctgagcgaggtggccacagggccagggcgggcgctgggtggagggcaggaggcaatgacactaatgctttttttttttttttttaactttttatctagaaattttatttttttaaacccggggcaagtacctcagctaactcccttcatcctcctggggcccctccttcctgccctcagtcttaggttagggccttggtcagggctttgctccctgtgacacccacaccctcgagctagcagcgtctcctcccttccccgggagagctggctggagacttggagctccgggaagtaggagtcacatttttttctctattctttggggattttttttacatgaataaaagtggatttcagggaaaaaaaaaaaaaaaaaaaaaaaaaaaaaa

SEQ ID NO: 32 - DNA, 23 bp, Artificial Sequence
  Description of Artificial Sequence Miscellaneous forward primer
cgactttgcctttccatttgctc

SEQ ID NO: 33 - DNA, 26 bp, Artificial Sequence
  Description of Artificial Sequence Miscellaneous reverse primer
ccttttgtgtttcatccttcctctcc

SEQ ID NO: 34 - DNA, 26 bp, Artificial Sequence
  Description of Artificial Sequence Miscellaneous forward primer
aaaggagaaagtgaaagatgtggagg

SEQ ID NO: 35 - DNA, 24 bp, Artificial Sequence
  Description of Artificial Sequence Miscellaneous reverse primer
ggacagaaagggaggacaggaaag

SEQ ID NO: 36 - DNA, 23 bp, Artificial Sequence
  Description of Artificial Sequence Miscellaneous forward primer
ccccacttcaaactctttcaccc

SEQ ID NO: 37 - DNA, 22 bp, Artificial Sequence
  Description of Artificial Sequence Miscellaneous reverse primer
gccatttcactgtcacgctttc

SEQ ID NO: 38 - DNA, 22 bp, Artificial Sequence
  Description of Artificial Sequence Miscellaneous forward primer
gctctgccaagacattgactcc

SEQ ID NO: 39 - DNA, 22 bp, Artificial Sequence
  Description of Artificial Sequence Miscellaneous reverse primer
atcatctcttccctctgcgtcc

SEQ ID NO: 40 - DNA, 24 bp, Artificial Sequence
  Description of Artificial Sequence Miscellaneous forward primer
cctacgtcactacactagagaccc

SEQ ID NO: 41 - DNA, 23 bp, Artificial Sequence
  Description of Artificial Sequence Miscellaneous reverse primer
gccaaaactgtctgcatactccc

SEQ ID NO: 42 - DNA, 22 bp, Artificial Sequence
  Description of Artificial Sequence Miscellaneous forward primer
aactgctcggtctatgtgcagc

SEQ ID NO: 43 - DNA, 24 bp, Artificial Sequence
  Description of Artificial Sequence Miscellaneous reverse primer
ccaagaacaccatgcagtacatcc

SEQ ID NO: 44 - DNA, 23 bp, Artificial Sequence
  Description of Artificial Sequence Miscellaneous forward primer
gctcattcaaaagaccgacaccg

SEQ ID NO: 45 - DNA, 22 bp, Artificial Sequence
  Description of Artificial Sequence Miscellaneous reverse primer
acacagttccatcagaccagcc

SEQ ID NO: 46 - DNA, 23 bp, Artificial Sequence
  Description of Artificial Sequence Miscellaneous forward primer
cgtctactgcctcaagagaaacc

SEQ ID NO: 47 - DNA, 24 bp, Artificial Sequence
  Description of Artificial Sequence Miscellaneous reverse primer
gtcctatgaccagagtcactctcc

SEQ ID NO: 48 - DNA, 23 bp, Artificial Sequence
  Description of Artificial Sequence Miscellaneous forward primer
aggaagaggaggaacagacagac

SEQ ID NO: 49 - DNA, 22 bp, Artificial Sequence
  Description of Artificial Sequence Miscellaneous reverse primer
agcagcctcaaaggacttgaac

SEQ ID NO: 50 - DNA, 22 bp, Artificial Sequence
  Description of Artificial Sequence Miscellaneous forward primer
aacctgctgctgatagaccacc

SEQ ID NO: 51 - DNA, 22 bp, Artificial Sequence
  Description of Artificial Sequence Miscellaneous reverse primer
tctctccactgctgcctgaaac

SEQ ID NO: 52 - DNA, 22 bp, Artificial Sequence
  Description of Artificial Sequence Miscellaneous forward primer
gtaagcaccagccacaaaaacc

SEQ ID NO: 53 - DNA, 23 bp, Artificial Sequence
  Description of Artificial Sequence Miscellaneous reverse primer
ctaacgagccattcccaataccc

SEQ ID NO: 54 - DNA, 22 bp, Artificial Sequence
  Description of Artificial Sequence Miscellaneous forward primer
tggattgggagatactgggcac

SEQ ID NO: 55 - DNA, 23 bp, Artificial Sequence
  Description of Artificial Sequence Miscellaneous reverse primer
ccaaacatcaggggaaccaaagg

SEQ ID NO: 56 - DNA, 22 bp, Artificial Sequence
  Description of Artificial Sequence Miscellaneous forward primer
cctgttcttcaacatgggccag

SEQ ID NO: 57 - DNA, 25 bp, Artificial Sequence
  Description of Artificial Sequence Miscellaneous reverse primer
cctctcaaccacctcctcaatcttc

SEQ ID NO: 58 - DNA, 26 bp, Artificial Sequence
  Description of Artificial Sequence Miscellaneous forward primer
tcttcttcccctaacatcaccatctc

SEQ ID NO: 59 - DNA, 22 bp, Artificial Sequence
  Description of Artificial Sequence Miscellaneous reverse primer
tgcatttgccagtcatgtcacc

SEQ ID NO: 60 - DNA, 24 bp, Artificial Sequence
  Description of Artificial Sequence Miscellaneous forward primer
aaaccctcttccttgtctcccctc

SEQ ID NO: 61 - DNA, 24 bp, Artificial Sequence
  Description of Artificial Sequence Miscellaneous reverse primer
atgtctgcttcttccccttgtgtc

SEQ ID NO: 62 - DNA, 22 bp, Artificial Sequence
  Description of Artificial Sequence Miscellaneous forward primer
tcaacaacaacccgaggaggag

SEQ ID NO: 63 - DNA, 22 bp, Artificial Sequence
  Description of Artificial Sequence Miscellaneous reverse primer
gatggcacagccaaagaggaag

SEQ ID NO: 64 - DNA, 23 bp, Artificial Sequence
  Description of Artificial Sequence Miscellaneous forward primer
acttccgcctcttcctgctaatc

SEQ ID NO: 65 - DNA, 24 bp, Artificial Sequence
  Description of Artificial Sequence Miscellaneous reverse primer
cctccaaaccatcttcatcttccc

SEQ ID NO: 66 - DNA, 24 bp, Artificial Sequence
  Description of Artificial Sequence Miscellaneous forward primer
atttcacagccccagttcacagcc

SEQ ID NO: 67 - DNA, 24 bp, Artificial Sequence
  Description of Artificial Sequence Miscellaneous reverse primer
tgaccacaatgaccaccactaccc

SEQ ID NO: 68 - DNA, 23 bp, Artificial Sequence
  Description of Artificial Sequence Miscellaneous forward primer
agcattaccagtacgtggggaag

SEQ ID NO: 69 - DNA, 24 bp, Artificial Sequence
  Description of Artificial Sequence Miscellaneous reverse primer
aacatactgccctccctgaggaac

SEQ ID NO: 70 - DNA, 24 bp, Artificial Sequence
  Description of Artificial Sequence Miscellaneous forward primer
taggctgtgagtcctgcaatgtcc

SEQ ID NO: 71 - DNA, 23 bp, Artificial Sequence
  Description of Artificial Sequence Miscellaneous reverse primer
tcagcatctcggcaagagtacac

SEQ ID NO: 72 - DNA, 24 bp, Artificial Sequence
  Description of Artificial Sequence Miscellaneous forward primer
aaccccaacaaggtccaggaacac

SEQ ID NO: 73 - DNA, 23 bp, Artificial Sequence
  Description of Artificial Sequence Miscellaneous reverse primer
tttccaccacaatggcgcaacag

SEQ ID NO: 74 - DNA, 20 bp, Artificial Sequence
  Description of Artificial Sequence Miscellaneous forward primer
aagttgcagtcttgcgtgtg

SEQ ID NO: 75 - DNA, 20 bp, Artificial Sequence
  Description of Artificial Sequence Miscellaneous reverse primer
ggtggttacctccttgtcca

SEQ ID NO: 76 - DNA, 22 bp, Artificial Sequence
  Description of Artificial Sequence Miscellaneous forward primer
cttgactgcttccctcaccaac

SEQ ID NO: 77 - DNA, 20 bp, Artificial Sequence
  Description of Artificial Sequence Miscellaneous reverse primer
cttttcacatgctgcacgcc

SEQ ID NO: 78 - DNA, 25 bp, Artificial Sequence
  Description of Artificial Sequence Miscellaneous forward primer
aggtggatgtgagggcaatgagaag

SEQ ID NO: 79 - DNA, 24 bp, Artificial Sequence
  Description of Artificial Sequence Miscellaneous reverse primer
cgggcgtgtagtaatgtgatgcag

SEQ ID NO: 80 - DNA, 24 bp, Artificial Sequence
  Description of Artificial Sequence Miscellaneous forward primer
gcctcctcttcgtcttttctaacc

SEQ ID NO: 81 - DNA, 25 bp, Artificial Sequence
  Description of Artificial Sequence Miscellaneous reverse primer
catcatcttgtgaaacaacagtgcc

SEQ ID NO: 82 - DNA, 22 bp, Artificial Sequence
  Description of Artificial Sequence Miscellaneous forward primer
tcaaggcatacccccttccaac

SEQ ID NO: 83 - DNA, 22 bp, Artificial Sequence
  Description of Artificial Sequence Miscellaneous reverse primer
agtccagtcaacacatcgctcc

SEQ ID NO: 84 - DNA, 23 bp, Artificial Sequence
  Description of Artificial Sequence Miscellaneous forward primer
tctatgctctttccccatacccc

SEQ ID NO: 85 - DNA, 22 bp, Artificial Sequence
  Description of Artificial Sequence Miscellaneous reverse primer
gcgatataccaggttgtgccag

SEQ ID NO: 86 - DNA, 24 bp, Artificial Sequence
  Description of Artificial Sequence Miscellaneous forward primer
gtgccaagtggaaaagttatgcag

SEQ ID NO: 87 - DNA, 24 bp, Artificial Sequence
  Description of Artificial Sequence Miscellaneous reverse primer
tgtcaacagatggacgaagacaag

SEQ ID NO: 88 - DNA, 23 bp, Artificial Sequence
  Description of Artificial Sequence Miscellaneous forward primer
ccccatttatcagctccattgcc

SEQ ID NO: 89 - DNA, 25 bp, Artificial Sequence
  Description of Artificial Sequence Miscellaneous reverse primer
catcccctcttctcacttcaacatc

SEQ ID NO: 90 - DNA, 22 bp, Artificial Sequence
  Description of Artificial Sequence Miscellaneous forward primer
ccaacctactgcaacttctgcc

SEQ ID NO: 91 - DNA, 22 bp, Artificial Sequence
  Description of Artificial Sequence Miscellaneous reverse primer
caaccccatcacactccaactc

SEQ ID NO: 92 - DNA, 22 bp, Artificial Sequence
  Description of Artificial Sequence Miscellaneous forward primer
gctctgccaagacattgactcc

SEQ ID NO: 93 - DNA, 22 bp, Artificial Sequence
  Description of Artificial Sequence Miscellaneous reverse primer
atcatctcttccctctgcgtcc

SEQ ID NO: 94 - DNA, 22 bp, Artificial Sequence
  Description of Artificial Sequence Miscellaneous forward primer
gttagccaagagccaggacaag

SEQ ID NO: 95 - DNA, 24 bp, Artificial Sequence
  Description of Artificial Sequence Miscellaneous reverse primer
gcaagccatatctgagaagccatc

(19) United States
(12) Patent Application Publication
(10) Pub. No.: US 2004/0110197 A1
(43) Pub. Date: Jun. 10, 2004

(54) Method of determining tumor characteristics by determining abnormal copy number or expression level of lipid-associated genes

(76) Inventors: Michael K. Skinner, Pullman, WA (US); Jodi L. Patton, Pullman, WA (US)

Correspondence Address: BAKER + HOSTETLER LLP, WASHINGTON SQUARE, SUITE 1100, 1050 CONNECTICUT AVE. N.W., WASHINGTON, DC, 20036-5304, US

(21) Appl. No.: 10/647,426

(22) Filed: Aug. 26, 2003

Related U.S. Application Data
(63) Continuation of application No. 09/676,052, filed on Sep. 28, 2000, now abandoned.

(51) Int. Cl. C12Q 1/68

(52) U.S. Cl. 435/6

(57) ABSTRACT

A method of assessing tumor characteristics in tissue samples by determining the copy number or expression level of genes associated with lipid metabolism, synthesis, or action is provided. Gene copy number may be assessed directly from chromosomal material or by determining the expression level of the gene in a tissue sample. The use of physical platforms comprising immobilized nucleic acid polymers to determine copy number or expression level of lipid associated genes by hybridization techniques is also provided.

### FIELD OF THE INVENTION

The present invention relates to a method of determining tumor characteristics in tissue samples taken from a patient by determining the copy number or expression level of genes associated with lipid metabolism, synthesis, or action in the sample. This determination may be made by directly quantifying the gene copy number in the chromosomal material of the tissue sample, or by determining the transcription level of the gene in the tissue sample. The present invention is also drawn to physical platforms which are useful in carrying out the diagnostic method, specifically arrays of nucleic acids useful for determining the copy number or expression level of the lipid associated genes by hybridization techniques.

### BACKGROUND OF THE INVENTION

Cancer is the second leading cause of death in the United States, after heart disease: one in every four Americans dies of cancer. As predicted by the SEER program at the National Cancer Institute, there will be over 1.2 million new cases of cancer diagnosed, and over a half-million cancer related deaths in the year 2000 alone. Cancer is characterized by several stages. First, there is an increase in the number of abnormal, or neoplastic, cells which arise from the normal cells in a tissue. Then, these neoplastic cells proliferate to form a tumor mass. Progression of the disease is then characterized by the growth of the tumor and the invasion of adjacent tissues by these neoplastic tumor cells. Finally, the tumor will generate malignant cells. which spread via the blood or lymphatic system to regional lymph nodes and to distant sites. The latter progression to malignancy is referred to as metastasis.

Cancer can be viewed as a breakdown in the cell growth regulatory system, in which proper communication between tumor cells, their normal neighboring cells, and the rest of the body is impaired. Signals, both growth-stimulatory and growth-inhibitory, are routinely exchanged between cells within a tissue. The maintenance of proper organ shape and function is dependant upon cell number stasis at particular positions in an organ. Normally, cells do not divide in the absence of stimulatory signals, and, likewise, will cease dividing in the presence of inhibitory signals. In a cancerous state, a cell bypasses this system of regulatory signals and proliferates under conditions in which normal cells would not.

Tumor cells must acquire a number of distinct aberrant traits to proliferate. Reflecting this requirement is the fact that the genomes of certain well-studied tumors carry several different independently altered genes, including activated oncogenes and inactivated tumor suppressor genes. Each of these genetic changes appears to be responsible for imparting some of the traits that, in aggregate, define a neoplastic cell (Land, H. et al., 1983, Science 222:771; Ruley, H. E., 1983, Nature 304:602; Hunter, T., 1991, Cell 64:249).

In addition to unhindered cell proliferation, cells must acquire several traits for tumor progression to occur. For example, early on in tumor progression, cells must evade the host immune system. Further, as tumor mass increases, the tumor must acquire vasculature to supply nourishment and remove metabolic waste. Additionally, cells must acquire an ability to invade adjacent tissue, and, ultimately, the capacity to metastasize to distant sites.

An increasing body of evidence implicates genetic mutations as causally important in the induction of human cancers. Advances in recombinant DNA technology have led to the discovery of normal cellular genes (proto-oncogenes and tumor suppressor genes) that control growth, development, and differentiation. Under certain circumstances, the regulation of these genes is altered, causing normal cells to assume neoplastic growth behavior. There are over 40 known proto-oncogenes and suppressor genes to date, which fall into various categories depending on their functional characteristics. These include, (1) growth factors and growth factor receptors, (2) messengers of intracellular signal transduction pathways, for example, between the cytoplasm and the nucleus, and (3) regulatory proteins influencing gene expression and DNA replication. Differential expression of the following suppressor genes has been demonstrated in human cancers: the retinoblastoma gene, RB; the Wilms' tumor gene, WT1 (11p); the gene deleted in colon carcinoma, DCC (18q); the neurofibromatosis type 1 gene, NF1 (17q); and the gene involved in familial adenomatous polyposis coli, APC (5q) (Vogelstein, B. and Kinzler, K. W., 1993, Trends Genet. 9:138-141).

Point mutations have been directly implicated in the causation of many human tumors. Some tumors carry oncogenes of the ras gene family, which differ from their normal cellular counterpart proto-oncogenes by the presence of a point mutation at one of a limited number of sites in these genes. Similarly, point mutations in critical regions of tumor suppressor genes, such as p53, are often detected in tumor cells. Mutation of the p53 suppressor gene is the most common alteration seen in epithelial tumors and, indeed, in all human tumors (Hollstein, M. et al., Science 253:49-53, 1991). When a tumor suppressor gene, such as p53, becomes mutated, cell proliferation accelerates in the absence of the suppressor. On the other hand, mutations in proto-oncogenes that transform them to active oncogenes, such as a mutant ras oncogene, produces cell proliferation caused by presence of the mutant gene itself.

The mutations that create active oncogenes have been explored with the hopes of providing important diagnostic and prognostic clues for tumor development. For example, a number of mutations have been found to alter the 12th codon of the ras oncogenes, causing replacement of a normally present glycine by any of a number of alternative amino acid residues. Such amino acid substitutions create a potent transforming allele. Thus, the presence of a particular nucleotide substitution may be a strong determinant of the behavior of the tumor cell (e.g., its rate of growth, invasiveness, etc.). As a result, nucleotide hybridization probes of oncogene mutations have been targeted for research as promising diagnostic reagents in clinical oncology.

In addition to point mutations, it has been shown that the amplification of single oncogenes can be linked to malignancy and proliferation of cancerous tumor cells. It is believed that many solid tumors, such as breast cancer, progress from initiation to metastasis through the accumulation of several such genetic aberrations. (Smith el: al., Breast Cancer Res. Treat., 18 Suppl. 1: S 514 (1991); van de Vijver and Nusse, Biochim. Biophys. Acta, 1072: 33-50 (1991); Sato et al., Cancer Res., 50: 71847189 (1990).) These genetic aberrations, as they accumulate, may confer proliferative advantages, genetic instability and the attendant ability to evolve drug resistance rapidly, and enhanced angiogenesis, proteolysis and metastasis. Deletions and recombination leading to loss of heterozygosity (LOH) are believed to play a major role in tumor progression by uncovering mutated tumor suppressor alleles.

Gene amplification is a common mechanism leading to upregulation of gene expression. (Stark et al., Cell. 75: 901-908 (1989).) Evidence from cytogenetic studies indicates that significant amplification occurs in over 50% of human breast cancers. (Saint-Ruf et al., supra.) A variety of oncogenes have been found to be amplified in human malignancies. Examples of the amplification of cellular oncogenes in human tumors are shown in Table 1 below.

TABLE 1

| | | Degree of |
|---|---|---|
| Amplified Gene | Tumor | Amplification |
| c-myc | Promyelocytic leukemia, cell | 20 x |
| | line, HL60 | |
| | Small-cell lung | 5-30 x |
| | carcinoma cell lines | |
| N-myc | Primary neuroblastomas | 5-1000 x |
| | (stages III and IV) and | |
| | neuroblastoma cell lines, | |
| | Retinoblastoma cell line and | 10-200 x |
| | primary tumors, | |
| | Small-cell lung carcinoma | 50 x |
| | cell lines and tumors | |
| L-myc | Small-cell lung carcinoma cell | 10-20 x |
| | lines and tumors | |
| c-myb | Acute myeloid leukemia | 5-10 x |
| | Colon carcinoma cell lines | 10 x |
| c-erbb | Epidermoid carcinoma cell | 30 x |
| | Primary gliomas | |
| c-K-ras-2 | Primary carcinomas of lung | 4-20 x |
| | colon, bladder, and rectum | |
| N-ras | Mammary carcinoma cell line | 5-10 x |

For example, as disclosed in U.S. Pat. No. 5,846,749, the amplification of the Her-2 gene has been linked to invasive breast cancer phenotypes. In a study of node-negative invasive breast carcinomas, the degree of HER-2/neu gene amplification was determined by Southern blot analysis of EcoRI digested tumor tissue and the relative amount of HER-2/neu mRNA was determined by Northern hybridization of total RNA The amount of HER-2/neu gene expression was roughly proportional to the number of copies of the gene in tumor cells. In addition, increasing levels of HER-2/neu gene amplification in these carcinomas were also associated with an increased risk of recurrent breast cancer. In human breast carcinomas, other proto-oncogenes, such as MYC, INT2, HST, and ERBB2, are frequently found either amplified or overexpressed. Although other similar correlations have been shown for other single oncogene amplifications or deletions, these approaches have proven to be or relatively limited predictive value for prognoses or treatment determinations.

Chromosomal deletions involving tumor suppressor genes may also play an important role in the development and progression of solid tumors. The retinoblastoma tumor suppressor gene (Rb-1), located in chromosome 13q14, is the most extensively characterized tumor suppressor gene (Friend et al., Nature, 323: 643 (1986); Lee et al., Science, 235: 1394 (1987); Fung et al., Science. 236: 1657 (1987)). The Rb-1 gene product, a 105 kDa nuclear phosphoprotein, apparently plays an important role in cell cycle regulation (Lee et al., supra (1987); Howe et al., PNAS (U.S.A.), 87: 5883 (1990)). Altered or lost expression of the Rb protein is caused by inactivation of both gene alleles either through a point mutation or a chromosomal deletion. Rb-1 gene alterations have been found to be present not only in retinoblastomas (Friend et al., supra (1986); Lee et al., supra (1987); Fung et al., supra (1987)) but also in other malignancies such as osteosarcomas (Friend et al., supra (1986)), small cell lung cancer (Hensel et al., Cancer Res., 50: 3067 (1990); Rygaard et al., Cancer Res., 50: 5312 (1990)) and breast cancer (Lee et al., Science, 241: 218 (1988); T'Ang et al., Science, 242: 263 (1988); Varley et al., Oncogene, 4: 725 (1989)).

In addition to single gene centered approaches to studying cancer genetics, changes in chromosomal loci associated with cancer have been studied. Chromosome abnormalities have long been associated with genetic disorders, degenerative diseases, and exposure to agents known to cause degenerative diseases, particularly cancer, German, “Studying Human Chromosomes Today,” American Scientist, 58: 182-201 (1970); Yunis; “The Chromosomal Basis of Human Neoplasia,” Science, 221: 227-236 (1983); and German, “Clinical Implication of Chromosome Breakage,” in Genetic Damage in Man Caused by Environmental Agents, Berg, Ed., pgs. 65-86 (Academic Press, New York, 1979). Chromosomal abnormalities include translocations (transfer of a piece from one chromosome onto another chromosome), dicentrics (chromosomes with two centromeres), inversions (reversal in polarity of a chromosomal segment), insertions, amplifications, and deletions.

In cancer, deletion or multiplication of copies of whole chromosomes or chromosomal segments, and higher level amplifications of specific regions of the genome, are common occurrences. With the advent of cloning and detailed molecular analysis, recurrent translocation sites have been recognized as involved in the formation of chimeric genes such as the BCR-ABL fusion in chronic myelogeneous leukemia. Deletions have been recognized as frequently indicating the location of tumor suppressor genes; and amplifications have been recognized as indicating overexpressed genes.

Human breast carcinomas are also characterized cytogenetically by various anomalies that may be the chromosomal counterpart of the molecular anomalies: regions of amplification are found in more than one-third of the tumors, and various deletions e.g., 1p, 11p, 11q, 13, and 17p, are found recurrently. Although amplification of genetic material is a frequent and probably important event in breast carcinogenesis, the relevant genes involved in such amplifications remain unknown, and do not seem to correspond to the proto-oncogenes commonly considered important in breast cancer. Since these regions of amplification in tumors are most often not at the site of the amplified genes in normal cells, standard cytogenetics does not yield any information that could assist with identification of the gene. Dutrillaux et al., Cancer Genet. Cytogenet., 49: 203-217 (1990) report (at page 203) that “(a)lthough human breast carcinomas are among the most frequent malignant tumors, cytogenetic data remain scarce, probably because of their great variability and of the frequent difficulty of their analysis.” In their study of “30 cases with relatively simple karyotypes to determine which anomalies occur the most frequently and, in particular, early during tumor progression” (p. 203), they concluded that “trisomy iq and monosomy 16q are early chromosomal changes in breast cancer, whereas other deletions and gain of 8q are clearly secondary events.” (Abstract, p. 203.) Dutrillaux et al. further state (at page 216) that deletions within tumor suppressor genes “characterize tumor progression of breast cancer.”

Restriction fragment length polymorphism (RFLP) studies have indicated that several tumor types have frequently lost heterozygosity at 13q, suggesting that one of the Rb-1 gene alleles has been lost due to a gross chromosomal deletion (Bowcock et al., Am. J. Hum. Genet., 46: 12 (1990)). The deletion of the short arm of chromosome 3 has been associated with several cancers, for example, small cell lung cancer, renal and ovarian cancers; it has been postulated that one or more putative tumor suppressor genes is or are located in the p region of chromosome 3 (ch. 3p) (Minna et al., Symposia on Ouantitative Biology, Vol. LI: 843-853 (SCH Lab 1986); Cohen et al., N. Eng. J. Med., 301: 592-595 (1979); Bergerham et al., Cancer Res., 49: 13901396 (1989); Whang-Peng et al., Can. Genet. Cytogenet., II: 91-106 (1984; and Trent et al., Can. Genet. Cytogenet., 14: 153-161 (1985)).

The Cancer Genome Anatomy Project, sponsored by the National Cancer Institute, has cataloged changes at particular chromosomal locations and correlated them with disease in the Breakpoint Map of Recurrent Chromosomal Aberrations. Unfortunately, the chromosomal change data consists of thousands of often conflicting anecdotal case studies, and chromosomal changes can only be mapped to large areas covering dozens of genes. Thus, although much data has been collected in this area, it has not yielded any discernable patterns in chromosomal change which are clinically useful for characterizing tumors or predicting the progress of cancers.

A universally accepted classification of cancer stages allows evaluation of treatment management, prognosis and statistical comparison for the various anatomic sites of cancer. Although several organizations dedicated to cancer exist, including the American Joint Committee on Cancer (AJCC), International Union Against Cancer (UICC); World Health Organization (WHO); Federation Internationale de Gynecologie et d'Obstertrique (FIGO), there has been a concerted effort to establish a uniform standard accepted world-wide for classification of cancer. In 1988, the AJCC in cooperation with the TNM Committee of the UICC accepted the TNM (Tumor, Node, and Metastasis) description to indicate the classification and stage of growth for the various anatomical sites of human cancer known. The AJCC Cancer Staging Manual (1997) is now in its 5^{th} edition, and corresponds with the Fifth Edition of the UICC TNM Classification of Malignant Tumors.

The TNM system is based on three significant events in the anatomical history of a cancer: the size of the untreated primary cancer (T), its spread to regional lymph nodes (N) and finally, distant metastasis beyond the regional lymph nodes (M). Classification of a cancer by TNM, therefore, indicates the extent of disease and progression for any cancer growth. Staging classifications are based on documentation of the anatomic extent of disease, derived from morphologic studies and clinical biopsies.

The AJCC, knowing that new information on diagnosis, treatment and etiology will affect the classification system, assigns task committees to meet periodically and recommend revisions. While now based mainly on anatomical criteria, it is very likely that molecular, genetic and other prognostic indicators will be included into the TNM classification system when recommended by the appropriate AJCC committee.

Clinical classification utilizing the TNM system is based on evidence acquired before primary treatment. Pathologic classification includes the evidence acquired before treatment, as well as evidence acquired from surgery. The three components, T, M and N, are assessed. The use of numerical subsets of the TNM components indicates the progressive extent of the malignant disease. Any of the T, N, or M classifications can be divided into subgroups for testing. The TNM components can then be evaluated to determine the stage grouping (I-IV) of growth for the patient's cancer. The clinical stage is used as a guide to the selection of primary therapy, usually a form of surgery to remove the cancerous lesions. The pathologic stage can also be used as a guide for adjuvant therapy (chemotherapy), prognosis, and reporting end results.

For example, over 90% of all ovarian cancers are derived from a single layer of surface epithelial cells that surrounds the ovary that are known as the ovarian surface epithelium (OSE). The cancerous epithelial cells initially form tumors on the ovary (stage I), slough off and migrate to the fallopian tubes and the uterus (stage II) and ultimately metastasize throughout the peritoneum and into such organs as the liver, bowel and bladder (stage III and IV). Stage I and II tumors are treatable with conventional management (up to 90% survival after five years for Stage IA and 70% survival for stage II), however, a lack of accurate diagnostics has made detecting early stage disease difficult with only 20% of all cases presenting at stage I or II. Most late stage ovarian cancer patients present with large volumes of ascites in the peritoneum. This accrual is thought to be a consequence of impaired lymphatic drainage as well as an increased rate of lymphatic production potentially due to effects of vascular permeability factor also known as vascular endothelial growth factor, which is present at high levels in ascites.

Treatment of all cancers depends on the tumor stage as determined by clinical evaluation and surgical resection. The standard technique for assessing the spread of a tumor is surgical resection of a primary tumor followed by careful review using light microscopy of surgical margins and other tissue, including lymph nodes. Under existing procedure, the adjacent tissue is stained by standard techniques and assessed under light microscopy for the presence of tumor cells. Using the TNM scale, the tumor can then be assessed and assigned to a particular stage. Accurate histopathologic assessment is critical since it provides important prognostic indicators that determine the probability of survival for a given patient following surgical resection of the primary tumor.

Despite many years of research and billions of dollars in expenditures, the long term survival of patients with malignancies remains disappointedly low, even where no tumor cells were detected in the tumor margins or more distant tissues. This inability to more accurately stage such patients might be due to the limitation inherent in the standard histopathologic methodology which is based upon visual observation and morphologic assessment under light microscopy of adjacent tissue and regional lymph nodes. Thus, a method which uses a more precise technique capable of determining spread of the disease at an earlier stage might provide a more accurate indication of the extent of tumor metastases into adjacent and regional tissues. Although limited advances have been made in the characterization of tumors and pre-cancerous tissues by studying changes in individual genes or at particular loci of chromosomes, these approaches do not take into consideration the interaction of various gene products which is necessary for the myriad steps of tumor progression. Thus, a need exists for a different approach towards the study of the progression of cancer and the development of better tumor diagnostic tools.

Bioactive lipids have recently been recognized to be an integral and pervasive part of cell regulation and signaling. Lysoglycerolphospholipids and sphingoid-based lipids are two important classes of bioactive lipid mediators that have been extensively studied in recent years and have known effects on ovarian cancer cells. LPA and lysophosphatidylcholine (LPC), as well as their sphingoid relatives, S1P and SPC along with the closely related platelet activating factor (PAF) can induce numerous cellular responses including cell proliferation, smooth muscle contraction, platelet aggregation, angiogenesis and tumor cell invasion. These mediators are produced by numerous cell lineages and are normal constituents of sera. Thus they can be viewed in terms of being a necessary part of physiological responses to specific cellular stresses. Furthermore, these lysophospholipids elicit their effects through classical signal transduction pathways including the regulation of heterotrimeric G-proteins, kinase signaling cascades, calcium mobilization, transcriptional regulation, focal adhesion kinase (p125 FAK) and the actin cytoskeleton. Over the past few years, considerable progress has been made in defining the molecular and cellular effects of LPA on cells, not the least of which is the cloning and characterization of several LPA receptors. These receptors are members of the seven-transmembrane-domain G-protein coupled receptor class and fall into two subfamilies: PSP24 and the Edg-family (endothelial cell differentiation gene) of receptors. The Edg-family is comprised of eight members of which Edg1, Edg2, Edg4 and Edg7 have been demonstrated to bind LPA and to activate a variety of signal transduction pathways in response to exogenous LPA. Edg1, Edg3, Edg5 and Edg6 have demonstrated to be receptors for S1P.

Phosphatidyl choline (PC), also named lecithin, is one of the major sources of polyunsaturated fatty acids such as arachidonic and linoleic acids. The former is a precursor of eicosanoids which have numerous biological activities. Hydrolysis of PC yields lysophosphatidyl choline (LPC) and constituent fatty acids, which have been implicated in signal transduction. An increasing body of evidence indicates that LPC, which is present in high concentrations in oxidized low density lipoproteins may play a significant role in atherogenesis and other inflammatory disorders. LPC has been reported to increase the transcription of genes encoding platelet derived growth factor A and B chains, and heparin-binding epidermal growth factor-like protein (HB-EGF) in cultured endothelial cells, and to increase mRNA encoding HB-EGF in human monocytes. These gene products are mitogens for smooth muscle cells and fibroblasts. LPC has also been shown to activate protein kinase C in vitro, to potentiate the activation of human T lymphocytes and to potentiate the differentiation of HL-60 cells to macrophages induced by either membrane-permeable diacylglycerols or phorbol esters.

LPC may also provide a source of bioactive lysophosphatidic acid (1-acyl-sn-glycero-3-phosphate, LPA) through hydrolysis by lysophospholipase D. LPA is a naturally occurring phospholipid with a wide range of growth factor-like biological activities. It is well established that LPA can act as a precursor of phospholipid biosynthesis in both eukaryotic and prokaryotic cells. The ability of LPA to act as an intercellular lipid mediator has been noted (Vogt, Arch. Pathol. Pharmakol. 240:124-139 (1960); Xu et al., J. Cell. Physiol. 163:441-450 (1995); Xu et al., Biochemistry 309:933-940 (1995); Tigyi et al., Cell Biol. 91:1908-1912 (1994); Panetti et al., J. Lab. Clin. Med. 129(2):208-216 (1997)). LPA is rapidly generated by activated platelets and can stimulate platelet aggregation and wound repair.

Ascites from ovarian cancer patients contain an activity or activities that are very effective at supporting the growth of ovarian cancer cell lines and of freshly isolated ovarian cancer cells but not of ovarian surface epithelial cells (OSE) or SV-40 large T-antigen-immortalized surface epithelial cells (IOSE). This growth factor-like activity in ascites (originally termed OCAF for Ovarian Cancer Activating Factor) was identified as lysophosphatidic acid (LPA) based on chemical and physical characteristics as well as the ability of synthetic LPA (but not other growth factors such as platelet-derived growth factor, also present in ascites) to completely desensitize the calcium mobilization response of ovarian cancer cells to ascites fluid and on the ability of phospholipases to destroy OCAF activity in ascites. (Mills et al., Cancer Res. 48:1066 (1988); Mills et al. J. Clin. Invest. 86:851 (1990) and U.S. Pat. Nos. 5,326,690 and 5,277,917)

LPA at concentrations present in ascites has multiple effects on ovarian cancer cells including increased cell proliferation, increased cell survival by decreasing apoptosis and anoikis, decreased sensitivity to cisplatin, increased invasiveness, increased production of vascular endothelial growth factor, increased production and activity of urokinase-type plasminogen activator (uPA) as well as the activity of the metalloproteinases MMP2 and MMP9 and finally, increased production of LPA itself. LPA does not produce similar responses in freshly isolated OSE and IOSE.

Other lysophospholipids associated with various conditions include lysophosphatidyl serine (LPS), lysophosphatidyl ethanolamine (LPE), lysophosphatidyl glycerol (LPG) and lysophosphatidyl inositol (LPI). Activated plate-lets secrete two kinds of phospholipase: sPLA2 and PS-PLA1. sPLA2 is reported to be elevated in inflammatory reactions and inhibition of this enzyme reduced inflammation. PS-PLA1 hydrolyzes phosphatidylserine or lysophosphatidyl serine (LPS) specifically to produce GPS or Glycerol-3-P serine. LPS strongly enhances degranulation of rat mast cells induced by concanavalin A and potentiates histamine release, and can stimulate sPLA2-elicited histamine release from rat serosal mast cells. LPS is an inflammatory lipid mediator and Spla2 has been implicated in inflammation processes. LPI has been shown to stimulate yeast adenylyl cyclase activity with implications for modulating the activity of downstream effector molecules and their interaction with RAS proteins.

Little is known about the mechanisms regulating LPA levels in vivo; however, the low LPA levels is plasma indicate that production, metabolism or clearance is tightly controlled. LPA is a normal phopholipid constituent of all cells and functions as a metabolic intermediate in de novo synthesis of glycerophospholipids and triglycerides. As with other lipid mediators like diacylglycerol and phosphoinositides, the relationship between this “housekeeping” LPA and LPA that exerts its actions through cell surface receptors in unclear. Clearly a growing variety of cells including platelets, adipocytes, leukocytes, fibroblasts, endothelial cells and, ovarian cancer cells, can release LPA into the extracellular space in response to agonist stimulation. Phospholipase A2 (PLA2)-mediated deacylation of phosphatidic acid (PA), produced by the action of phospholipase D (PLD) on membrane phosphatidylcholine (PC) or by the actions of diacylglycerol kinase on diacylglycerol formed by phospholipase C likely contributes to LPA production in response to cellular activation. The pathway for production of extracellular LPA has been most intensely studied in platelets where release of membraneous microvesicles is a critical step.

### SUMMARY OF THE INVENTION

Although the role of bioactive lipids in the proper functioning of an organism is not fully understood, their varied biological activities indicate that they are important cell regulation and signaling molecules. As mentioned above, elevated LPA levels have been linked to ovarian cancer, and several sphingolipids and lysophospholipids have been implicated in cell signaling events. Applicants postulated that these altered lysophospholipid and sphingolipid levels were indicative of a loss of control over proteins involved in lipid metabolism, synthesis, and signaling at a genomic level, and that this loss of control over bioactive lipid signaling functions is an integral event in the progress of cancer development. Using this unique metabolic approach, applicants have developed a novel method of monitoring cancer progression. The added information concerning tumor characteristics that is supplied to the clinical practitioner by this method can be used to choose more appropriate and effective cancer therapies.

The present invention is drawn to a method for identifying tumor characteristics in tissue samples taken from a patient by determining the copy number or expression level of genes associated with lipid metabolism, synthesis, or action in the sample. Although not bound to any particular theory, applicants believe that the alteration of the chromosomal copy number or loss of control over the expression of lipid associated genes is a very significant event in the genesis and progression of cancer. Thus, the relative stage and characteristics of the disease may be monitored by determining the copy number or expression level of lipid associated genes in the cancerous and pre-cancerous cells of a tumor or surrounding tissues. This determination may be made by directly quantifying the gene copy number in the chromosomal material of the tissue sample, or by determining the transcription level of the gene in the tissue sample. The present invention is also drawn to physical platforms which are useful in carrying out the diagnostic method, specifically arrays of nucleic acids useful for determining the copy number or expression level of the lipid associated genes by hybridization techniques.

Thus, one aspect of the present invention is a method for identifying tumor characteristics from a tissue sample obtained from a patient, wherein the method comprises determining whether the cells of the tissue sample have an abnormal copy number or expression level of at least two genes associated with lipid metabolism, synthesis, or action. Preferred genes for monitoring in making the determination are Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, EDG 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Triacylglycerol lipase, EDG 2, EDG 3, EDG 4, EDG 5, EDG 6, and EDG 7. Particularly preferred genes for monitoring in the method of the invention are Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, EDG 1, Glycerol-3-phosphate dehydrogenase, EDG 2, EDG 3, EDG 4, EDG 5, EDG 6, and EDG 7.

Although useful information concerning tumor characteristics may be gleaned by determining the copy number or expression level of as few as two lipid associated genes, it is preferred that more than two genes be so monitored in the methods of the invention. The copy number or expression level of each gene can usually be used to answer one question about the tumor characteristics of the tissue sample, such as “Is the cell metastatic?” or “Is the tumor at stage 2?” Furthermore, where the source of the tumor is not known with complete certainty, the copy number or expression level of each gene can indicate the source of the tumor and provide important information about the origin or source of the tumor and yield insight into the diagnosis or treatment thereof. If more lipid associated genes are monitored, more information will be available to the clinician. In addition, the correlation between tumor stages and the amplification or deletion of any particular gene is usually not 100%. Thus, certainty as to the tumor characteristics identified through the method can be increased by monitoring more than one lipid associated gene that has been correlated to a particular characteristic, or “redundant” genes. Thus, methods employing at least three, four, five, six, seven, eight, nine, ten, fifteen, or twenty genes are all more preferred embodiments of the method of the invention. A most preferable embodiment of the method would determine the copy number or expression level of at least ten lipid associated genes, as this will provide several pieces of information concerning the stage and metastatic potential of the cells in the tissue sample with redundant genes to verify that information.

The method of the invention may be applied to a tissue sample from any organ, including muscle, dermal tissues, lung, liver, pancreas, stomach, colon/rectal tissues, bone, prostate, testicles, or other organs in which cancer is known to occur. However, especially preferred embodiments of the invention utilize tissue samples from gynecological organs, including breast, cervix, uterus, and ovaries. It is also contemplated that connective, muscle, or lymphoid tissues surrounding the organ of interest will also be a source of tissue samples, as it may be advantageous for a clinician to test these tissues for evidence of invading or metastasizing tumor cells.

The determination of whether the cells of the tissue sample have an abnormal copy number or expression level of the genes associated with lipid metabolism, synthesis, or action may be achieved by any of several methods known in the art for determining gene copy number or expression level. For example, in situ fluorescence hybridization techniques can be used to microscopically “count” the number of gene copies in a cell. Preferred methods for determining the copy number or expression level of lipid associated genes are those which use hybridization probes. Such methods generally include the steps of:

a) isolating sample nucleic acid polymers from the cells of the tissue sample;

b) hybridizing the sample nucleic acid polymers with nucleic acid polymers specific for the selected genes under conditions wherein the extent of hybridization may be quantified; and

c) comparing the hybridization data thus obtained with data obtained from the hybridization of reference nucleic acid polymers isolated from a normal cell of the same tissue type as that of the tissue sample from the patient with the nucleic acid polymers specific for the selected genes under the same conditions.

In the methods of the present invention, the sample nucleic acid polymers and the reference nucleic acid polymers may be either genomic DNA or mRNA encoding expressed genes. It is also contemplated to be within the scope of the claimed method to amplify the sample nucleic acid polymer by a polymerase chain reaction (PCR) technique prior to hybridization in order to increase the amount of the sample nucleic acid polymer available for detection.

The nucleic acid polymer probes specific for the selected genes used in the methods or arrays of the invention are preferably derived from the naturally occurring gene sequence of the gene for which the probe is designed. It is preferable that the nucleic acid probe comprise at least about 19 nucleotides which hybridize under the hybridization conditions used in the method to a similarly sized portion of the naturally occurring gene sequence. However, it is more preferable to use nucleic acid probes with at least about 25 nucleic acids which hybridize to the naturally occurring gene, so that allelic variations and point mutations will not significantly interfere with the detection of the lipid associated gene copy number or expression level. The nucleic acid probe may hybridize with a coding region of one of the selected genes, or with a non-coding sequence functionally linked to the coding region of one of the selected genes, wherein the functionally linked sequence is unique to that gene.

It is also preferred in embodiments of the methods of the present invention to immobilize the nucleic acid polymer probes specific for the selected genes on a solid support, so that a nucleic acid polymer specific for each selected gene is located at a different predetermined position on the solid support. When the nucleic acid polymer probes are so arranged, the copy number or expression level of several lipid associated genes in the sample nucleic acid polymers may be determined at the same time by sandwich hybridization assay techniques.

Thus, another aspect of the present invention is an array of nucleic acid polymers immobilized on a solid support, in which the array comprises:

a) a solid support;

b) at least two different nucleic acid polymers which are each specific for a different gene associated with lipid metabolism, synthesis, or action; wherein a nucleic acid polymer specific for each gene associated with lipid metabolism, synthesis, or action is located at a different predetermined position on the solid support, and wherein the array comprises less than 100 nucleic acid polymers which are specific for genes other than the selected genes. Although the arrays of the present invention may be part of a larger array for testing nucleic acid polymers isolated from a tissue sample, and such embodiments are envisioned as within the scope of the invention, the arrays are nonetheless intended to be used as relatively focused tools for gathering information about tumor characteristics. Thus, arrays which contain nucleic acid polymer probe sequences specific for each and every human gene, or those which consist of probe sequences specific for every gene expressed by a certain cell type (also referred to as “cDNA library arrays), are not considered to be within the scope of the present invention.

It is preferred that at least two of the nucleic acid polymers which are specific for genes associated with lipid metabolism, synthesis, or action be specific for genes selected from the group consisting of: Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, EDG 1, Glycerol-3-phosphate dehydrogenase, Sphingosine-1 -phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Triacylglycerol lipase, EDG 2, EDG 3, EDG 4, EDG 5, EDG 6, and EDG 7. It is more preferred that at least one of the nucleic acid polymers which is specific for a gene associated with lipid metabolism, synthesis, or action is specific for a gene selected from the group consisting of: Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, EDG 1, Glycerol-3-phosphate dehydrogenase, EDG2, EDG3, EDG4, EDG5, EDG6, and EDG 7.

### DESCRIPTION OF FIGURES AND SEQUENCE ID'S

FIG. 1: A diagram of the pathways for various metabolism and synthesis of phospholipids.

FIG. 2: A diagram of the pathways for various metabolism and synthesis of sphingolipids.

SEQ ID NO. 1: The sequence of the cDNA coding for 1 -acylglycerol-3-phosphate acyltransferase.

SEQ ID NO. 2: The sequence of the cDNA coding for Aldehyde dehydrogenase (5 family, member A1).

SEQ ID NO. 3: The sequence of the cDNA coding for Choline/ethanolamine phosphotransferase.

SEQ ID NO. 4: The sequence of the cDNA coding for Diacylglycerol kinase, gamma.

SEQ ID NO. 5: The sequence of the cDNA coding for Dihydroxyacetone phosphate acyltransferase.

SEQ ID NO. 6: The sequence of the cDNA coding for EDG 1.

SEQ ID NO. 7: The sequence of the cDNA coding for EDG 2.

SEQ ID NO. 8: The sequence of the cDNA coding for EDG-3.

SEQ ID NO. 9: The sequence of the cDNA coding for EDG-4.

SEQ ID NO. 10: The sequence of the cDNA coding for EDG-5.

SEQ ID NO. 11: The sequence of the cDNA coding for EDG-6.

SEQ ID NO. 12: The sequence of the cDNA coding for EDG-7.

SEQ ID NO. 13: The sequence of the cDNA coding for Glycerol-3-phosphate dehydrogenase.

SEQ ID NO. 14: The sequence of the cDNA coding for Lyosphospholipase I.

SEQ ID NO. 15: The sequence of the cDNA coding for Human Lysophospholipase Homolog.

SEQ ID NO. 16: The sequence of the cDNA coding for N-acylsphingosine amidohydrolase.

SEQ ID NO. 17: The sequence of the cDNA coding for Phospholipase A2.

SEQ ID NO. 18: The sequence of the cDNA coding for Phospholipase D1 (phosphatidylcholine specific).

SEQ ID NO. 19: The sequence of the cDNA coding for Phospholipase D1 glycosylphosphatidylinositol specific.

SEQ ID NO. 20: The sequence of the cDNA coding for Phosphatidic Acid Phosphatase type 2b.

SEQ ID NO. 21: The sequence of the cDNA coding for Phosphatidic Acid Phosphatase type 2a.

SEQ ID NO. 22: The sequence of the cDNA coding for Phosphatidylinositol-3-Kinase (class2, gamma polypeptide).

SEQ ID NO. 23: The sequence of the cDNA coding for Phosphatidylinositol-3-kinase (catalytic, alpha polypeptide).

SEQ ID NO. 24: The sequence of the cDNA coding for Prostate Differentiation Factor PLAB.

SEQ ID NO. 25: The sequence of the cDNA coding for Phosphatidic Acid Phosphatase type 2c.

SEQ ID NO. 26: The sequence of the cDNA coding for Phosphocholine cytidyltransferase.

SEQ ID NO. 27: The sequence of the cDNA coding for Phosphate cytidylyltransferase 2 (ethanolamine specific).

SEQ ID NO. 28: The sequence of the cDNA coding for Phosphatase and Tenson Homolog (PTEN).

SEQ ID NO. 29: The sequence of the cDNA coding for Sphingosine-1-phosphate lyase 1.

SEQ ID NO. 30: The sequence of the cDNA coding for Sphingomyelin phosphodiesterase 1.

SEQ ID NO. 31: The sequence of the cDNA coding for Phospholipase C beta 3 (phosphatidylinositol specific).

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention is directed to a method of identifying tumor characteristics by determining the copy number or expression level of genes associated with lipid metabolism, synthesis, or action in the cells of a tissue sample from a patient, and to arrays used as a tool in the claimed methods. Although the examples and guidance given below primarily concern embodiments of the invention which include the use of array-based nucleic acid hybridization techniques, one of ordinary skill in the art will readily recognize that the invention is not limited to this particular type of methodology for determining lipid- associated gene copy number or expression level. Thus, the method of identifying tumor characteristics taught by the present invention can be readily modified by those of skill in the art to utilize any other acceptable methods of determining gene copy number or expression level in the cells of a sample, and such modifications are considered to be within the scope of the present invention.

### DEFINITIONS

As used herein, “tumor characteristic” means any morphological characteristic associated with tumors or the development and progression of cancers, such as tumor stage and serous, mucinous, metastatic, or endometroid character. Especially, The metastatic nature and potential of the tumor cells, or the degree of their invasive and migratory nature, may be determined by particular lipid-associated gene copy number or expression level profiles. Tumor stage, defined according to traditional pathological criterion, is of particular interest as a tumor characteristic. Although the traditional methods of describing tumor stages (size, degree of invasion into regional tissues, and the presence of distant metastasis (or colonies of cells from the original tumor)) are not directly determined by the present method, the underlying biochemical changes which allow the tumor cells to drastically increase in size, to live detached from the tumor surface, and to infiltrate the cell layers of surrounding tissues are often linked to changes in the copy number or expression level of lipid-associated genes. In addition, resistance to particular chemotherapies or radiation may be determined from the lipid-associated gene copy number or expression level profile, as the success of particular therapies often depends upon the cell characteristics of the tumor.

“Tissue sample” means any tissue obtained from a patient which is suspected of comprising cancer or tumor cells, including tissues from the tumor body, tissues surrounding the tumor, or tissues obtained from vicinal lymph nodes.

“Patient,” as used herein, is not limited exclusively to human patients. However, the exemplary methods described herein are intended for use with human patients. Modification of the methods described herein, particularly with respect to the particular genes assayed for copy number or expression level, are probably necessary for application of these methods to non-human patients. Several genes associated with lipid metabolism, synthesis, or action are known for a number of non-human mammalian species, and the modification of the present methods for use with these species would be well within the skill of the ordinary practitioner in the biochemical arts.

“Abnormal copy number,” as used herein, denotes a higher or lower number of copies of a gene in a cell's genome, as compared to the normal number of gene copies in a cell of a particular type in a particular organism. Although ordinarily two copies of most genes are contained in most diploid cells, greater or fewer copies may be considered “normal” for the cell. For instance, only one copy of some genes on the X chromosome is present in a normal diploid cell from a male organism (XY heterozygous).

“Expression level,” as used herein, denotes the level of transcription of a gene in a cell, as determined by the number of mRNA's encoding a particular protein gene product present in the cell. The number of mRNA copies of most genes will vary by cell tissue type. Thus, an abnormal expression level would be one in which a significantly higher or lower number of mRNA's for a particular protein exist in the cell as compared to a normal cell of the same tissue type under approximately the same conditions.

37 Genes associated with lipid metabolism, synthesis, or action,” include genes whose proteins modify, oxidize, reduce, cleave, bind, or otherwise utilize bioactive lipids as substrates or ligands. Such bioactive lipids include sphingosine-1-phosphate (S1P), sphingophosphatidylcholine (SPC), sphingophosphatidylinositol (SPI), sphingophosphatidylserine (SPS), shingophosphatidylglycerol (SPG), sphingophosphatidylethanolamine (SPE), lysophosphatidic acid (LPA), lysophatidylcholine (LPC), lysophosphatidylserine (LPS), lysophosphatidylinositol (LPI), lysophosphatidyl ethanolamine (LPE), and lysophosphatidyl glycerol (LPG), as well as metabolites of these lipids such as glycerol-3-phosphate (G3P). A non-exhaustive list of such genes include those encoding Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Sphingosine kinase, Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1 -acylglycerol-3-phosphate acyltransferase, Phosphatidic Acid Phosphate type 2b, EDG 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Glycerol-3-phosphate Acyltransferase, Diacylglycerol Lipase, Phosphatidylethanolamine methyltransferase, Ceramide cholinephosphotransferase, N-acylsphingosine glucosyltransferase, Sphingosine N-acyltransferase, Triacylglycerol lipase, Phosphatidylserine decarboxylase, CDP Diacylglycerol inositol-3-phosphatidyltransferase, EDG 2, EDG 3, EDG 4, EDG 5, EDG 6, and EDG 7.

The term “isolating nucleic acid polymers” means any acceptable method of extracting nucleic acid polymers (such as deoxyribonucleic acids or ribonucleic acids) from the cells of a sample. Normally, such methods will include steps such as lysing the cells in a sample and purifying the nucleic acids from the lysed cells. Traditional methods of purification include solvent partition of cell components or absorption of nucleic acids onto a matrix which has a high affinity for nucleic acids, such as silica. However, “isolation” of the nucleic acid in a sample, as the term is used in this application, can also be accomplished by fixing the nucleic acid within the cell at a known or knowable location, such as in paraffin fixed tissues whose chromosomal DNA can be examined microscopically by microkeratotomy and histopathology techniques. These methods are well known to those of ordinary skill in the art.

The term “hybridizing” denotes a procedure in which complementary, or nearly complementary, nucleic acid polymers are allowed to associate in solution. Usually, if a sample nucleic acid polymer is double stranded chromosomal DNA, the hybridization procedure will include a denaturation or “melting” step. It is preferred that buffers and temperature conditions in the hybridization procedure be such that minimal non-specific interactions between the associating nucleic acid polymers occur. These are usually referred to as “stringent” conditions by those of skill in the art. This will prevent non-specific binding of the probe nucleic acid polymer to the sample nucleic acid polymers. However, a slight tolerance for mismatched base pair association is also preferable in a hybridization procedure for use in the present invention. This will account for minor allelic variations in the human genetic make-up and for small numbers of substitution mutations (a not uncommon occurrence in tumor cell genomes.) Thus, the conditions used in the hybridization procedure will preferably allow approximately one mismatched base pair association per 15-20 associated based pairs. Hybridization conditions and methods are well known in the art, and it is within the capabilities of one of ordinary skill in the art to adjust such methods to allow for the preferred level of stringency.

The term “conditions wherein the extent of hybridization may be quantified” means any accepted method for quantifying the amount of nucleic acid polymers derived from a sample tissue which associate specifically with the nucleic acid polymer probe used to measure the copy number of the gene of interest. Many methods have been disclosed in the art for making such a quantitative determination. They range from in situ fluorescence hybridization using fluorescently labeled nucleic acid polymer probes to visualize gene copy number on interphase chromosomes (such a system specific for the HER2 gene is offered by Vysis, Downers Grove, Ill., USA), to electrically enhanced hybridization systems with fluorescently labeled probes (such as that described in U.S. Pat. No. 6,017,696.) Preferred methods for use in the present invention are those which utilize arrays of nucleic acid probed in which different populations of nucleic acid probes are affixed to predetermined locations in the array. Such arrays allow for the hybridization of the sample nucleic acid polymers with several different probes specific for different genes at the same time.

The term “a normal cell of the same tissue type” means a non-cancerous, non-tumor cell of the same tissue type of the tumor's originating cells, or of the surrounding tissue from which the tissue sample is taken. For instance, a normal cell for comparison with a ovarian tumor tissue sample would be obtained from a healthy ovary.

The term “amplified” in the context of sample nucleic acid polymers means that the nucleic acid polymer is replicated in order to obtain a detectable amount of the nucleic acid polymer. Usually, such amplification is effected by self-replicating the nucleic acid polymer through the polymerase chain reaction. Methods of utilizing DNA and RNA polymerases to amplify sample nucleic acid polymers are well known to those of ordinary skill in the biological arts. When amplifying nucleic acids for use in the methods of the present invention, it is preferable that care be taken to normalize the data obtained from the amplified samples in order to obtain quantifiable results. Such a normalization may be achieved by subjecting a substantially similar amount of the reference nucleic acid polymers to the same or similar amplification procedure and obtaining hybridization data to compare to the hybridization data obtained with the amplified sample nucleic acid polymers.

The term “coding region” means a region of the gene which is translated into the amino acid sequence of the gene product, including signal peptides or portions of a pro-peptide which are cleaved from the gene product in order to form a mature protein. Generally, such regions are termed expressed sequences, or “exons.”

The term “non-coding sequence functionally linked to the coding regions of the gene” means nucleic acid sequences which are normally not translated into the amino acid sequence of the gene product, but nevertheless form a part of the functional gene. Such sequences include introns, promoters, enhancers, and nucleic acid sequences which fill in between these elements of the gene and the coding regions of the gene.

The term “immobilized on a solid support” means that the nucleic acid polymer is bound, covalently or though an affinity reaction, to a relatively contiguous surface. The solid support may consist of any appropriate material for binding nucleic acids, including glass, silicas, hydrogels (such as agarose or polyacrylamide), polymers (such as polystyrene or polypropylene), and cellulose derivatives (such as nitrocellulose). The solid support may be in any convenient form for quantifying the amount of hybridization, including beads, resins, microtiter wells, flat surfaces, rods, and the like. For use in the arrays of the present invention, flat silicate surfaces, such as used in U.S. Pat. No. 5,744,305, are preferred. Immobilization formats adapted to these surfaces have been developed which can be easily loaded with the lipid associated gene probe sequences described in the invention, and which can be easily read with automated equipment.

The term “predetermined position” means that the location of immobilization on the solid support is known, and can be determined by reference to a detectable orientation point on or attached to the solid support. It is preferred that the surface of the solid support be divided into a grid-like arrangement of positions, in which a different population of nucleic acid polymers specific for a different gene resides at each position, or an “array”. Although it is preferred that this array be arranged in a symmetrical and orderly fashion, such an arrangement is not necessary. The only requirement is that the position of the particular populations of nucleic acid polymers specific for each gene be identifiable. In this way, the hybridization of the sample nucleic polymers to a particular population of nucleic acid polymers in the array may be determined by the physical location of that hybridization, as visualized by fluorescence or detected by some other means.

The term “specific,” as applied to nucleic acid polymers, means that the nucleic acid polymer probe contains a sequence substantially complementary to the lipid associated gene of interest, and not to other genes. Normally, a nucleic acid polymer will be specific for a gene if the probe sequence comprises at least about 19 contiguous nucleotides which are identical or complementary to 19 contiguous nucleotides of the lipid associated gene of interest. It is preferred that the nucleic acid polymer probe contain at least 1-5 additional contiguous identical or complementary nucleic acids: this will ensure hybridization under stringent conditions if a substitution mutation or allelic variation in the sample nucleic acid polymers is present. Those of ordinary skill in the art will recognize that the nucleic acid polymer probes may contain further nucleic acids in order to provide an anchor for immobilizing the nucleic acid polymer on a solid support or for attaching a detectable label to the nucleic acid polymer probe.

### Choice of Lipid-Associated Genes for Copy Number of Expression Level Determination

The first step in carrying out the methods of the invention is choosing which lipid-associated genes to monitor in the tissue samples. Genes associated with lipid metabolism, synthesis, or action suitable for monitoring in the present invention include genes whose proteins modify, oxidize, reduce, cleave, bind, or otherwise utilize bioactive lipids as substrates or ligands. Such bioactive lipids include sphingosine-1-phosphate (S1P), sphingophosphatidylcholine (SPC), sphingophosphatidylinositol (SPI), sphingophosphatidylserine (SPS), sphingophosphatidylglycerol (SPG), sphingophosphatidylethanolamine (SPE), lysophosphatidic acid (LPA), lysophatidylcholine (LPC), lysophosphatidylserine (LPS), lysophosphatidylinositol (LPI), lysophosphatidyl ethanolamine (LPE), and lysophosphatidyl glycerol (LPG), as well as metabolites of these lipids such as glycerol-3-phosphate (G3P). Several of the biological metabolic pathways involving these bioactive lipids and the enzymes involved are illustrated in FIGS. 2 and 3. A non-exhaustive list of genes associated with lipid metabolism, synthesis, or action suitable for monitoring in the present invention include those encoding Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Sphingosine kinase, Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, EDG 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Glycerol-3-phosphate Acyltransferase, Diacylglycerol Lipase, Phosphatidylethanolamine methyltransferase, Ceramide cholinephosphotransferase, N-acylsphingosine glucosyltransferase, Sphingosine N-acyltransferase, Triacylglycerol lipase, Phosphatidylserine decarboxylase, CDP Diacylglycerol inositol-3-phosphatidyltransferase, EDG 2, EDG 3, EDG 4, EDG 5, EDG 6, and EDG 7. The copy number or expression level of at least two lipid-associated genes is monitored in the methods of the present invention. Most preferably, at least one of the genes monitored is chosen from the group consisting of Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, EDG 1, Glycerol-3-phosphate dehydrogenase, EDG 2, EDG 3, EDG 4, EDG 5, EDG 6, and EDG 7.

Usually, by monitoring the copy number or expression level of a particular gene one will be able to answer at most one question about the tumor characteristics of the tissue sample, such as “Is the cell metastatic?” or “Is the tumor at stage 2?”. If more lipid associated genes are monitored, more information will be available to the clinician. Thus, although useful information concerning tumor characteristics may be gleaned by determining the copy number or expression level of as few as two lipid associated genes, it is preferred that more than two genes be so monitored in the methods of the invention. Preferably, a sufficient number of genes to fully characterize the tumor are monitored in the methods of the invention. Thus, complex profiles of increased and decreased lipid-associated gene copy number or expression level for all known lipid-associated genes may be established for various tumor stages and characteristics. However, simplified versions of the present method may be useful in some instances. For example, where a particularly strong correlation exists between metastasis and an increase in gene copy number or expression level, for instance in the Phosphatidylinositol-3-kinase (alpha polypeptide) and Phospholipase D1 (phosphatidylcholine specific) genes (See data in Table 2), a clinician may choose to only assay the copy number of those particular genes in order to gather information about the metastatic potential of the cells. This situation could arise when a breast tumor biopsy specimen is being evaluated histologically, and an in-situ hybridization format for gene copy number counting is preferred. Even this limited amount of information can be very useful in assisting a physician in counseling a patient as to whether a breast-conserving lumpectomy or mastectomy is indicated.

As illustrated in Tables 2 and 3, several lipid-associated genes are present in altered copy numbers in significant numbers of ovarian cancer tumors. % loss and gain was determined by chromosomal genomic hybridization. These data indicate that the genes associated with lipid metabolism, synthesis, and action play an important role in tumor development and cancer progression. Altered numbers of these genes in tumor cells allow drastically increased (or decreased) production of these critical gene products, and thereby profoundly influence the morphology and physiology of the tumor cell.

As discussed previously, it is usually better to monitor several genes which have been correlated to the same tumor characteristic (metastatic, serous, mucinous, stage 3, etc.) at the same time. As indicated in the illustrative data collected in Tables 2 and 3, the correlation between the altered copy number or expression level of a particular gene and particular tumor characteristics is usually not 100%. Although not bound by any particular theory, applicants believe that this is due to the fact that several alternative pathways exist for the synthesis and metabolism of bioactive lipid products, as is illustrated in FIGS. 2 and 3. Thus, loss of control over bioactive lipid synthesis at any one of a number of points can lead to particular tumor characteristics such as vascularization or the ability to grow detached from the tumor surface (a necessary condition for metastasis). Thus, several alternative genetic changes exist which will lead to the same phenotypic characteristic. Therefore, in order to more accurately determine the characteristics of a particular tumor for diagnostic and case management purposes, it is preferable to monitor several lipid-associated genes simultaneously in order to gather “redundant” information about a particular tumor characteristic. Thus, methods employing at least three, four, five, six, seven, eight, nine, ten, fifteen, or twenty genes are all more preferred embodiments of the method of the invention. A most preferable embodiment of the method would determine the copy number or expression level of at least ten lipid associated genes, as this will provide several pieces of information concerning the stage and metastatic potential of the cells in the tissue sample with redundant genes to verify that information.

### Construction of Nucleic Acid Polymer Probes and Primers Specific for the Chosen Liped Associated Genes

Once the lipid-associated genes to be monitored have been chosen, probes may be designed to assay the copy number or expression level of those genes in a tissue sample according to any number of conventional methods, including hybridization to immobilized arrays, dot-blot hybridizations, southern blot techniques, or even chromosomal counting through fluorescent in-situ hybridization. The choice of probe and probe production technique will depend on the method used.

For instance, solid-support based synthesis techniques are favored for the production of short probes for immobilization or to use as labeled in-situ hybridization probes. Techniques for synthesizing short oligonucleotides include conventional phosphotriester and phosphodiester methods or automated derivatives thereof. In one such automated method, diethylphosphoramidites are used as starting materials and may be synthesized as described by Beaucage, et al. (Tetrahedron Letters, 22:1-1862, 1981). Another method for synthesizing oligonucleotides on a modified solid support is described in U.S. Pat. No. 4,458,066. DNA solid support synthesis is a routine matter for one of skill in the biochemical arts, and several commercial services currently exist which will synthesize DNA sequences of up to 500 bp in length, and which may include fluorescent, biotin, or hapten labeled nucleotides (e.g., Genset of La Jolla, Calif.). Thus, once designed, labeled or unlabeled short DNA probes may simply be purchased from a vendor.

Shorter (under 500 bp) nucleic acid polymer probes specific for the lipid associated genes may be designed according to general principles familiar to those of ordinary skill in the biochemical arts. Usually, it is preferable to use a probe containing at least 19 contiguous base pairs which are identical or complementary to 19 contiguous base pairs of the naturally occurring sequence in the lipid-associated gene. Thus, any portion of the sequences SEQ ID NO. 1-31 larger than 19 base pairs would be suitable for use as a lipid-associated gene probe in the present invention. This number of bases allows for hybridization under relatively stringent conditions, and also contains enough sequence information to ensure that the sequence is usually unique to that particular lipid-associated gene. Of course, one of ordinary skill in the art would understand that common enhancer sequences, polyadenylation sequences, and other sequences which tend to be repeated amongst several mammalian genes should not be used as probe sequences for the lipid-associated genes.

In order to ensure that the probe will hybridize to the lipid-associated gene DNA in the sample if some allelic variation or mutation difference exists between the probe sequence and the sample DNA sequence, it is more preferable that the probe sequence include at least 1-5 additional nucleotides which are identical or complementary to the nucleotide sequence of one of the lipid associated genes, or 20-24 contiguous nucleotides which are identical or complementary to the nucleotide sequence of one of the lipid associated genes. This will account for a small amount of mismatching during hybridization. Due to the prominent role of point mutations in oncogenesis, accounting for such slight variations is preferred in order to correctly assess the copy number or expression level of the lipid-associated genes to be assayed.

The maximum length of the probes to be used will be ascertainable by one of ordinary skill in the art based upon such considerations as the propensity for the formation of secondary structures in the probe DNA, the likelihood of interactions between probe strands in an immobilized matrix embodiment, and the difficulties in reproducing DNA of certain lengths through methods such as the polymerase chain reaction. Although full length gene sequences, including all introns, exons, regulatory sequences, and intervening DNA, may be used as lipid-associated gene probes, such sequences are often several dozen kilobases in length. Sequences of this length have a propensity to form secondary structures and are difficult to replicate by polymerase chain reaction techniques (which is often a necessary step in the production of longer sequence probes). Thus, probe sequences shorter than the full length gene are preferred as lipid-associated gene probes. Suitable longer probes would include whole exons, or multiple exons as found in a cDNA sequence. One of ordinary skill in the art would be able to choose a suitable long portion of any of SEQ ID NO. 1-31 for use as a long lipid-associated gene probe. Such probes are produced in Example 1.

In order to produce a DNA probe sequence longer than 500 bp, it is preferable to amplify and clone the sequence from a cDNA or genomic library according to methods familiar to those of ordinary skill in the molecular biology arts. Briefly, one synthesizes short (˜20-25 nucleotide) forward and reverse polynucleotide primers which flank the portion of the lipid-associated gene's sequence to be cloned. Then, one amplifies the sequence from a cDNA library or genomic DNA utilizing the polymerase chain reaction technique, as described below for amplification of sample nucleic acids. Once a sufficient amount of amplified DNA sequence has been produced, it may be ligated into a suitable plasmid vector, and replicated/maintained in a bacterial host. The DNA sequence may then be cut out of the plasmid with suitable restriction enzymes and either labeled or immobilized for use as a lipid-associated gene probe.

Probes which are to be immobilized in an array are not labeled for detection. Rather, they are covalently linked to a predetermined position in the array, and thus identifiable by location. In these embodiments, the sample DNA to be hybridized with the array is usually labeled utilizing labeled primers during PCR amplification, or by other techniques known in the art. If the probe is to be hybridized to immobilized sample DNA (as in, for example, dot-blot hybridization, the reporter probe in a sandwich hybridization, or in-situ hybridization), then the probe itself is labeled for detection.

Labeling

The particular label or detectable group attached to the probe or primer nucleic acids is not a critical aspect of the invention, so long as it does not significantly interfere with the hybridization of the probe to the lipid-associated gene sequence in the sample DNA. The detectable group can be any material having a detectable physical or chemical property. Such detectable labels have been well-developed in the field of nucleic acid hybridizations and in general most any label useful in such methods can be applied to the present invention. Thus a label is any composition detectable by spectroscopic, photochemical, biochemical, immunochemical, electrical, optical or chemical means. Useful labels in the present invention include fluorescent dyes (e.g., fluorescein isothiocyanate, texas red, rhodamine, and the like) radiolabels (e.g., ³H, ¹²⁵I, ³⁵S, ¹⁴C, or ³²p), and enzymes (e.g., horse radish peroxidase, alkaline phosphatase and others commonly used in an ELISA).

The nucleic acids can be indirectly labeled using ligands for which detectable anti-ligands are available. For example, biotinylated nucleic acids can be detected using labeled avidin or streptavidin according to techniques well known in the art. In addition, antigenic or haptenic molecules can be detected using labeled antisera or monoclonal antibodies. For example, N-acetoxy-N-2-acetylaminofluorene-labelled or digoxigenin-labelled probes can be detected using antibodies specifically immunoreactive with these compounds (e.g., FITC-labeled sheep anti-digoxigenin antibody (Boehringer Mannheim)). In addition, labeled antibodies to thymidine-thymidine dimers can be used (Nakane et al. ACTA Histochem. Cytochem. 20:229 (1987)).

Generally, labels which are detectable in as low a copy number as possible, thereby maximizing the sensitivity of the assay are preferred. A label is preferably chosen that provides a localized signal, thereby providing spatial resolution of the signal from each probe. The labels may be coupled to the DNA in a variety of means known to those of skill in the art. In a preferred embodiment the probe or sample DNA will be labeled using nick translation or random primer extension (Rigby, et al. J. Mol. Biol., 113:237 (1977) or Sambrook, et al., Molecular Cloning—A Laboratory Manual, Cold Spring Harbor Laboratory, Cold Spring Harbor, N.Y. (1985)).

### Construction of Immobilized Nucelic Acid Polymer Probe Arrays

In preferred embodiments of the invention, nucleic acid polymer probes specific for the chosen lipid-associated genes are immobilized on a solid surface in order to create an array of lipid-associated-gene probes. As discussed above, the solid surface may be in any desirable shape which facilitates the hybridization reaction and the detection of hybridized sample nucleic acids. The solid support may be in any convenient form for quantifying the amount of hybridization, including beads, resins, microtiter wells, flat surfaces, rods, and the like. Illustrative solid materials for use in the support include nitrocellulose, nylon, glass, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cements or the like. In addition substances that form gels can be used. Such materials include proteins (e.g., gelatins), lipopolysaccharides, silicates, agarose and polyacrylamides. Where the solid surface is porous, various pore sizes may be employed depending upon the nature of the system. For use in the arrays of the present invention, flat coated or uncoated silicate surfaces are preferred. Immobilization formats adapted to these surfaces have been developed which can be easily loaded with the lipid associated gene sequences described in the invention, and which can be easily read with automated equipment.

In preparing the solid support for binding the nucleic acid polymer probes to it, several different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, enhance signal detection or the like. If covalent bonding between the probe and support is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various supports is well known and is amply illustrated in the literature. For example, methods for immobilizing nucleic acids by introduction of various functional groups to the molecules is known (see, e.g., Bischoff et al., Anal. Biochem. 164:336-344 (1987); Kremsky et al., Nuc. Acids Res. 15:2891-2910 (1987)). In order to introduce these functional groups, modified nucleotides (such as biotin, halogen, or azo-derivatized nucleotides) can be placed on the nucleic acid probe using PCR primers containing the modified nucleotide, or by enzymatic end labeling with modified nucleotides.

Use of membrane supports (e.g., nitrocellulose, nylon, polypropylene) for the nucleic acid arrays of the invention is advantageous because of well developed technology employing manual and robotic methods of arraying nucleic acid probes at relatively high element densities (e.g., up to 30-40/cm²) while maintaining each probe population as a distinct element. In addition, such membranes are generally available and protocols and equipment for hybridization to membranes is well known. Many membrane materials, however, have considerable fluorescence emission, where fluorescent labels are used to detect hybridization. To optimize a given assay format one of skill can determine sensitivity of fluorescence detection for different combinations of membrane type, fluorochrome, excitation and emission bands, spot size and the like. In addition, low fluorescence background membranes have been described (see, e.g., Chu et al., Electrophoresis 13:105-114 (1992)).

Arrays on substrates with much lower fluorescence than membranes, such as glass, quartz, or silicon, can achieve much better sensitivity. For example, elements of various sizes, ranging from the˜1 mm diameter down to˜1 μm can be used with these materials. Small array members containing small amounts of concentrated target DNA are conveniently used for the methods of the invention since the total amount of sample DNA available for binding to each probe will be limited. Thus it is advantageous to have small array members that contain a small amount of concentrated probe DNA so that the signal that is obtained is highly localized and bright. Such small array members are typically used in arrays with densities greater than 10⁴/cm². Relatively simple approaches capable of quantitative fluorescent imaging of 1 cm² areas have been described that permit acquisition of data from a large number of members in a single image (see, e.g., Wittrup et. al. Cytometry 16:206-213 (1994)).

Covalent attachment of the target nucleic acids to glass or synthetic fused silica can be accomplished according to a number of known techniques. Such substrates provide a very low fluorescence substrate, and a highly efficient hybridization environment. There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized. Recently, several refined techniques have been developed to array nucleic acid polymers on silicon or coated silicon surfaces, such as those disclosed in U.S. Pat. Nos. 5,143,854 and 6,017,696. These methods, which utilize photomasking or electrostatic techniques to couple nucleic acid polymers at specific sites on the support, are able to achieve high concentrations of each nucleic acid polymer probe at specific locations on the silicon support. These techniques are preferred for creating the arrays of the present invention, as the resulting arrays can be readily utilized in automated hybridization and fluorescence detection equipment.

The nucleic acid probes can also be immobilized on commercially available coated beads or other supports. For instance, biotin end-labeled nucleic acids can be bound to commercially available avidin-coated beads or a layer of avidin-permeated hydrogel. Streptavidin or anti-digoxigenin antibody can also be attached to silanized glass slides by protein-mediated coupling using e.g., protein A following standard protocols (see, e.g., Smith et al. Science, 258:1122-1126 (1992)). Biotin or digoxigenin end-labeled nucleic acids can be prepared according to standard techniques.

Any of these techniques can be utilized by the person of ordinary skill in the art to create an array of probes specific for lipid-assoicated genes for use in the present invention. An illustrative array, utilizing probes generated from the cDNA sequences of several lipid-associated genes, is constructed on a silanized glass slide in Example 1.

### Tissue Sample Preparation and Nucleic Acid Isolation

In order to carry out the method of identifying tumor characteristics of the present invention, it is necessary to obtain a tissue sample from the patient. Several standard techniques for biopsy of tissue samples are well known in the prior art. Preferably, the tissue samples are obtained by endoscopic biopsy, or by a biopsy needle gun. When obtaining a tissue sample from a solid tumor in the patient, care should be taken not to allow portions of the tumor to escape from the tumor body into the other tissues of the patient.

The tissue which is isolated can be used directly, frozen, or it can be embedded in, for example, paraffin and stored for future use. Preferably, the tissue is frozen and stored at temperatures of −20° to about −80° C. The term “embedded” refers to a sample that has been infiltrated with a material to provide mechanical support and thereby reduce sample deformation during processes such as sectioning (preparing thin slices for viewing using a microscope). Embedding materials include waxes, such as paraffin wax, epoxies, gelatin, methacrylate, nitrocellulose, various polymers and the like. The term “non-embedded” refers to a sample that is not embedded, and was not previously embedded. When a tissue sample is embedded in, for example, paraffin, for future use, it will preferably be in sections of about 6 micron thickness. Upon removal from storage, the paraffin-embedded tissue sections will be deparaffinized using a relatively non-polar aprotic organic solvent such as xylene, and then rehydrated using graded alcohols followed by phosphate-buffered saline (PBS). Other suitable solvents for removing the embedding support include aliphatic or aromatic hydrocarbon solvents such as toluene, heptanes, octanes, benzene, acetone and acetonitrile. If a technique such as in situ fluorescent hybridization is used to determine the copy number of the lipid-associated genes, then the preparation of the embedded tissue sample should be carried out according to methods known in the art which maintain the fixed position of the sample nucleic acids in the cell.

If the tissue sample is to be analyzed by an array hybridization technique, or another technique which requires purified isolated nucleic acids, the cells of the tissue sample may be treated to liberate and isolate their nucleic acid polymers. For lysing, chemical lysing will conveniently be employed utilizing detergents or cell membrane degrading enzymes. Several methods of isolating nucleic acids from lysed cells are well known in the art, such phenol extraction followed by ethanol or polyethylene glycol precipitation, or the ketone method disclosed in U.S. Pat. No. 5,063,162. Several kits are commercially available to perform genomic DNA or RNA extractions on tissue samples.

Often, the hybridization data obtained from the sample nucleic acid polymers will be compared to that of reference nucleic acid polymers obtained from normal tissues of the same type as the tissue sample. In order to facilitate accurate comparison, the normal tissue sample should be obtained in a similar method as the tissue sample to be tested, and the reference nucleic acids should be isolated from the normal tissue sample in the same manner as the nucleic acids from the patient's sample are isolated. Practitioners of ordinary skill in the art are capable of devising such “controls” for a quantitative comparison based on the specific nucleic acid isolation and hybridization methods which are to be used in any particular embodiment of the method of the invention.

Amplification of Sample Nucleic Acid Polymers

In practicing the methods of the present invention, it is often preferable to amplify the sample nucleic acid polymer sequence before hybridization in order to increase the hybridization signal. The tissue samples obtained from biopsy are often very small in size, and contain a limited amount of sample nucleic acids for detection. In addition to increasing the amount of DNA or RNA available to produce a hybridization signal, amplification reactions may be used to label the sample nucleic acids by utilizing radiolabled, biotinylated, or fluorescent-moiety substituted nucleic acids or primers. Amplification of the sample nucleic acid polymers can be accomplished using oligonucleotide primers for amplification specifically designed for the lipid-associated gene to be assayed.

These unique oligonucleotide primers are based upon identification of the flanking regions contiguous with the nucleotide sequence of the lipid-associated genes chosen for copy number or expression level determination. In this manner, it is possible to selectively amplify the specific target nucleic acid sequence containing the nucleic acid of interest. For example, the flanking sequence of the primer may be before the gene in a normal genome, a complementary sequence (opposite strand) after the gene in the normal genome, or within the gene itself. The only requirement of the primer is that it hybridize at a position that will ensure that the portion of the gene for which the nucleic acid probe has been designed will be amplified. As most amplification reactions do not produce nucleic acid products of more than a few hundred base pairs, it is preferred that the primer hybridize about 10-100 bases before the probe hybridization site. One of ordinary skill in the art would be capable of designing an appropriate primer for the amplification of any lipid-associated gene monitored, given the information supplied in SEQ ID NO. 1-31, and other available genetic information concerning lipid-associated genes.

Experimental conditions conducive to synthesis include the presence of nucleoside triphosphates and an agent for polymerization, such as DNA polymerase, and a suitable temperature and pH. Preferably the reaction mixture will contain labeled nucleotides which can be used to detect the amplified sample nucleic acid polymers when bound to nucleic acid probes in an array. Any of the nucleotide derivatives described in the probe construction section above are suitable for use in the amplification reaction. The use of fluorescent derivatives of nucleic acids to label the sample nucleic acid polymers is particularly preferred in the embodiments of the method of the invention. The primer is preferably single stranded for maximum efficiency in amplification, but may be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent for polymerization. The exact length of primer will depend on many factors, including temperature, buffer, and nucleotide composition.

Typically, the amplification process is started by annealing two primers to the sample nucleic acid polymers: one primer is complementary to the negative (−) strand of the nucleotide sequence and the other is complementary to the positive (+) strand. Annealing the primers to denatured nucleic acid followed by extension with an enzyme, such as the large fragment of DNA Polymerase I (Klenow) or Taq DNA polymerase (or an RNA reverse-transcriptase, if the mRNA of a sample is to be amplified) and nucleotides or ligases, results in newly synthesized + and − strands containing the target nucleic acid. Because these newly synthesized nucleic acids are also templates, repeated cycles of denaturing, primer annealing, and extension results in exponential production of the region (i.e., the target mutant nucleotide sequence) defined by the primer. The product of the amplification reaction is a discrete nucleic acid duplex with termini corresponding to the ends of the specific primers employed. Those of skill in the art will know of other amplification methodologies which can also be utilized to increase the amount of the sample nucleic acid polymers and label them for later detection. One method of amplification which can be used according to this invention is the polymerase chain reaction (PCR) described in U.S. Pat. Nos. 4,683,202 and 4,683,195.

### Hybridization Techniques

The particular hybridization technique used will depend on the particular embodiment of the method of the invention. For instance, if the copy number of the lipid-associated genes is to be determined using in-situ gene counting techniques, then hybridization of labeled probes with fixed sample tissues on a slide would be performed according to techniques known in the art. Preferably, the copy number or expression level of the lipid-associated gene sequences in the sample nucleic acids is determined by hybridizing the labeled sample nucleic acids to an array of lipid-associated gene specific probes. The hybridization signal intensity produced by the labeled sample nucleic acids on each lipid-associated gene probe site is determined. Typically, the greater the signal intensity at a particular lipid-associated gene probe site, the greater the copy number or expression level of the lipid-associated gene sequence in the sample nucleic acid. Thus, a determination of the signal intensity at each probe site, as compared to a normal tissue standard, allows a determination of the copy number or expression level of the lipid-associated gene.

Standard hybridization techniques are used to probe the lipid-associated gene probe array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). In addition, several refinements of hybridization techniques relevant to microarray hybridization analysis are discussed in Khan, et al., “Expression Profiling in Cancer Using cDNA Microarrays,” Electrophoresis, 20:223-229 (1999); and Cheung, et al., “Making and Reading Microarrays,” Nature Genetics Supp., 21: 15-19 (1999).

Generally, nucleic acid hybridizations comprise the following major steps: (1) immobilization of the probe nucleic acids; (2) prehybridization treatment to increase accessibility of the immobilized DNA, and to reduce nonspecific binding; (3) hybridization of the sample nucleic acids to the probe nucleic acid on the solid support; (4) posthybridization washes to remove sample nucleic acid fragments not bound in the hybridization and (5) detection of the hybridized sample nucleic acid fragments. The reagents used in each of these steps and their conditions for use vary depending on the particular embodiment of the method of the present invention.

Pre-hybridization may be accomplished by incubating the microarray with the hybridization solution at room temperature or at a mildly elevated temperature for a sufficient time to thoroughly wet the array. Usually, incubation at about 35° to 42° C. for about 15 minutes to an hour is sufficient to pre-hybridize a microarray on a glass slide.

Various hybridization solutions may be employed, comprising from about 20% to 60% volume, preferably 30%, of an inert polar organic solvent. A common hybridization solution employs about 50% formamide, about 0.5 to 1M sodium chloride, about 0.05 to 0.1M sodium citrate, about 0.05 to 0.2% sodium dodecylsulfate, and minor amounts of Denhardt's solution (EDTA, ficoll (about 300-500 kD), polyvinylpyrrolidone, (about 250-500 kD) and serum albumin.) Optionally, one may include other blocking agents in the hybridization solution, such as about 0.5 to 5 mg/ml of sonicated denatured DNA, e.g., calf thymus or salmon sperm; and optionally from about 0.5 to 2% wt/vol glycine. Other additives may also be included, such as dextran sulfate of from about 100 to 1,000 kD and in an amount of from about 8 to 15 weight percent of the hybridization solution, or a similarly sized polyethylene glycol.

Various degrees of stringency of hybridization may be employed in the methods of the invention. The more severe the conditions, the greater the complementarily that is required for hybridization between the sample nucleic acids and the lipid-associated gene specific probe for duplex formation. Severity can be controlled by temperature, probe concentration, probe length, ionic strength, time, and the like. Conveniently, the stringency of hybridization is varied by changing the polarity of the reactant solution by manipulating the concentration of formamide in the range of 20% to 50%. Temperatures employed will normally be in the range of about 20.degree. C. to 80.degree. C., usually 30.degree. C. to 75.degree. C. (see, generally, Current Protocols in Molecular Biology, Ausubel, ed., Wiley & Sons, 1989).

After the array has been contacted with a hybridization solution at a moderate temperature for a period of time sufficient to allow hybridization to occur (usually 8-24 hours), the filter is then introduced into a second solution having similar concentrations of sodium chloride, sodium citrate and sodium dodecylsulfate as provided in the hybridization solution. The time the filter is maintained in the second solution may vary from five minutes to three hours or more. The second solution determines the stringency, dissolving cross duplexes and short complementary sequences. After rinsing the filter at room temperature with dilute sodium citrate-sodium chloride solution, the filter may now be assayed for the presence of hybridized sample nucleic acids according to the nature of the label.

### Analysis of Detectable Signals from Hybridization of the Sample Nucleid Acids with the Lipid-Associated Gene Probes

Standard methods for detection and analysis of signals generated by the hybridization of the sample nucleic acids to the lipid-associated gene specific probes can be used. The particular methods will depend upon the labels used, and the particular embodiment of the method. Generally, fluorescent labels are preferred. Thus, methods suitable in fluorescence in situ hybridization (FISH) are suitable in the present invention. The nucleic acid arrays are imaged in a fluorescence microscope with a polychromatic beam-splitter to avoid color-dependent image shifts. The different color images are acquired with a CCD camera and the digitized images are stored in a computer.

If the preferred microarray embodiments are utilized, such fluorescence data is conveniently gathered with an automated array scanner, such as the Affymetrix 418 Array scanner with epi-fluorescent confocal optics. A computer program is then used to analyze the signals produced by the array in order to determine the hybridization intensity at each lipid-associated gene probe site in the array. This data may then be compared with a standard curve generated from various amounts of DNA or mRNA obtained from normal tissue. This analysis technique is illustrated in Example 2.

The present invention is further described in the following examples. These examples do not, in any way, limit the present invention.

### EXAMPLES

The following examples illustrate the preparation of the arrays of lipid-associated genes of the invention for use in the methods of the invention for determining tumor characteristics. The molecular biological techniques utilized in these examples are well-known to those of ordinary skill in the biochemical arts, and could be modified in myriad ways to accomplish the same ends. Thus, the techniques and processes described below serve merely to illustrate an embodiment of the present invention, and should not be interpreted to limit the invention to any particular embodiment. Unless otherwise noted, all kits, reagents, and equipment were used in accordance with the protocols supplied by the producer. More detailed protocols for the techniques described in brief below may be found in a standard molecular biology reference text, such as Maniatis.

### Example 1

### Preparation of a Lipid-Associated Gene Probe Array

In this example, the preparation of a hybridization microarray of 31 probes for lipid-associated genes on a standard glass slide is prepared for use in the methods of the invention.

1.1 Preparation of DNA Probes for Use in the Microarray

For each probe to be used in the array, an appropriate pair of unlabeled forward and reverse PCR primers (listed in Table 4), synthesized utilizing solid-support methods, is ordered from Genset (La Jolla, Calif.). Using these primers, DNA for each probe is amplified by PCR from a human genomic library (available from Invitrogen, Carlsbad, Calif., Stratagene, La Jolla, Calif., or Promega, Madison, Wis.) with Taq polymerase (obtained from Promega, Madison, Wis.) according to supplier's instructions. PCR is carried out in an Eppendorf Scientific Instruments Mastercycler™ thermal cycler for the recommended number of cycles. After PCR, the amplified DNA (which contains single adenine overhangs) is self-ligated into the AccepTor™ plasmid (Novagen, Madison, Wis.) and transformed into NovaBlue E. coli competent cells (Novagen).

Transformed cells are plated onto LB blue/white selection media and grown overnight. White colonies (which contain the amplified DNA insert) are picked, and grown overnight for freezer stocks. After verification of the DNA insert, a 1 liter culture of a clone containing the insert is prepared for each probe, and plasmid DNA is purified from the culture using a Wizard® Plus Megaprep DNA Purification System (Promega). The plasmid DNA is then digested with EcoR I (Promega), and the insert-probe DNA separated from the AccepTor™ plasmid DNA by agarose gel electrophoresis. Probe size in the agarose gel is double checked against DNA size standards to ensure that no cleavage with the restriction enzyme occurs. The probe DNA is then purified from the agarose gel slices and resuspended in 2×SSC (Saline Sodium Citrate) buffer at a concentration of about 1 μg/μl for aminosilane linkage to the glass slide substrate of the array.

1.2 Production of the Microarray

The probes are affixed to a glass slide using the aminosilane linkage chemistry described in Cheung, et al., “Making and Reading Microarrays,” Nature Genetics Supp., 21:15-19 (1999). The 31 probes are arrayed in a 6 by 5 plus 1 pattern on a CMT-GAPS™ aminosilane coated slide (Corning, Acton, Mass.). The assymetrical 6 by 5 plus 1 design of the lipid-associated probe array facilitates rapid identification of each spot in the array. Approximately 10 ng of each probe DNA is deposited in spots approximately 125 μm in diameter and about 300 μm apart (from center to center). Probes are arrayed using an Affymetrix 417 Arrayer (Affymetrix, Santa Clara, Calif.) according to manufacturer's instructions.

The arrayed probes are allowed to air-dry on the slide at room temperature. The slides are then briefly moistened in hot water vapor before crosslinking the DNA to the silane surface with about 0.30 J/cm² of ultraviolet (254 nm) radiation. After crosslinking, the slides are briefly washed in 0.1% SDS to remove unbound DNA. The probes are then denatured in 95° C. water for approximately three minutes.

The resulting microarray of lipid-associated gene probes is then ready for use in sample testing.

### Example 2

### Determination of Lipid-Associated Gene Copy Number in a Sample Utilizing the Lipid Associated Gene Probe Array

In this example, the copy number of several lipid-associated genes is determined simultaneously by hybridization to the probe produced in Example 1.

2.1 Preparation of Sample DNA

Genomic DNA is extracted from approximately 300 mg of sample tissue using a Wizard® Genomic DNA Purification Kit (Promega). After purification, the concentration of sample DNA is determined using spectraphotometric techniques. 40 μg of sample is then amplified by PCR with a mixture of fluorescein labeled primers for all lipid-associated genes monitored by the array (all of those listed in Table 4, obtained from Genset) using Taq polymerase (obtained from Promega) according to supplier's instructions. PCR is carried out in an Eppendorf Scientific Instruments Mastercycler™ thermal cycler for the recommended number of cycles. After PCR, the amplified sample DNA is separated from the labeled primers with a Wizard™ PCR Preps DNA Purification System (Promega). Purified sample DNA is then resuspended in 15 μl of hybridization solution (50% formamide, 6×SSC, 0.5% SDS, 5×Denhardt's reagent).

2.2 Hybridization of Sample DNA to the Array

The microarray is pre-hybridized with hybridization solution for about 30 minutes. The sample DNAs are denatured for about 4 minutes at 80° C. in a hybridization oven. The amplified sample DNA in the hybridization solution is then applied to the microarray in a Corning CMT™ Hybridization Chamber, and then allowed to anneal at 42° C. for about 20 hours. After hybridization has been completed, the array slide is washed for five minutes in 0.1% SDS/0.2×SSC, and then for about five minutes with 0.2×SSC. The array is then loaded into and Affymetrix 418 Scanner (Affymetrix), and fluorescence of the hybridized sample DNA on the microarray is detected.

2.3 Data Analysis and Determination of Lipid Associated Gene Copy Number

A standard curve is generated for each probe site in the array utilizing the above procedure with 20 μg (haploid), 40 μg (normal), 80 μg (tetraploid), 160 μg (octaploid), and 320 μg (hexadecaploid) of genomic DNA purified from normal tissue. By comparison of detected fluorescent intensity from the hybridization of the sample DNA at each probe site with the standard curve for that probe site, the relative copy number of the lipid-associated genes in the cells of the tissue sample is calculated.

### Example 3

### Demonstration of the Correlation Between Lipid-Associated Gene Copy Number and Tumor Characteristics

The correlation between lipid-associated gene copy number and certain tumor characteristics is demonstrated by determining the copy number of lipid-associated genes according to the method described in Example 2, utilizing a lipid-associated gene probe array produced as described in Example 1.

Tumor tissue samples from stage 1, 2, 3, and 4 tumors have been assayed, as well as those characterized as “serous,” “mucinous,” “endometroid,” “low stage,” and “high stage.” Tumors from various types of cancers, including ovarian, breast, cervical, and uterine are analyzed, normal tissues of each type provide a control and a standard curve. This analysis demonstrates that specific tumor stages and characteristics correlate to increases or decreases in the copy number of certain lipid-associated genes.

TABLE 2
Enzymes Involved with Lipid Metabolism
Chromosome Location With Associated Ovarian Tumor Stage and Clinical Outcome

| | | | Associated Tumor | Associated Clinical |
|---|---|---|---|---|
| Enzyme | % loss | % gain | Stage | Outcome |
| Phosphatidylinositol-3-Kinase, catalytic, | 0 | 50 | Low and High | Reduced Survival |
| alpha polypeptide | | | Stage | Duration |
| Phospholipase D1 phosphatidylcholine | 0 | 40 | Low and High | Reduced Survival |
| specific | | | Stage | Duration |
| Sphingosine kinase | −5 | 20 | Low and High | |
| | | | Stage | |
| Aldehyde dehydrogenase 5 family, member | −5 | 15 | Borderline tumors | |
| A1 | | | | |
| Phospholipase D1 | −5 | 15 | Borderline tumors | |
| glycosylphosphatidylinositol specific | | | | |
| 1-acylglycerol-3-phosphate acyltransferase | −5 | 15 | Borderline tumors | |
| Sphingosine-1-phosphate lyase 1 | 0 | 5 | Low Stage - | |
| | | | Endometroid | |
| Phosphatase and tenson homolog (PTEN) | −2.5 | 5 | Low Stage - | |
| | | | Endometroid | |
| Phosphatidic Acid Phosphate type 2a | −2.5 | 2.5 | Borderline tumors | |
| Sphingomyelin phosphodiesterase 1 (acid | −5 | 0 | High Stage - | Reduced Survival |
| sphingomyelinase) | | | nonmucinous | Duration |

TABLE 3
Enzymes Involved with Lipid Metabolism
Chromosomal Locations and Associated Genome Copy Number
Changes in Ovarian Tumors

| Enzyme | % loss | % gain |
|---|---|---|
| Phosphatidylinositol-3-Kinase, catalytic, | 0 | 50 |
| alpha polypeptide | | |
| Phosphate cytidylytransferase 1, choline, | 0 | 50 |
| alpha isoform | | |
| Phospholipase D1 phosphatidylcholine | 0 | 40 |
| specific | | |
| dihydroxyacetone phosphate acyltransferase | 0 | 22.5 |
| Phosphate cytidylytransferase 2, ethanolamine | −2.5 | 20 |
| Sphingosine kinase | −5 | 20 |
| Phosphatidic Acid Phosphate type 2c | 0 | 20 |
| Prostate differentiation factor PLAB | 0 | 20 |
| (associated with Edg 3) | | |
| Phospholipase A2 | −2.5 | 17.5 |
| Phospholipase C, beta 3 (phosphatidylinositol | −5 | 17.5 |
| specific) | | |
| Phosphatidylinositol-3-Kinase, class 2, | 0 | 17.5 |
| gamma polypeptide | | |
| Choline/ethanolamine phosphotransferase | −2.5 | 15 |
| Lysophospholipase | 0 | 15 |
| Aldehyde dehydrogenase 5 family, member | −5 | 15 |
| A1 | | |
| Phospholipase D1 | −5 | 15 |
| glycosylphosphatidylinositol specific | | |
| 1-acylglycerol-3-phosphate acyltransferase | −5 | 15 |
| Phosphatidic Acid Phosphate type 2b | −2.5 | 7.5 |
| EDG 1 | −2.5 | 7.5 |
| Glycerol-3-phosphate dehydrogenase | 0 | 5 |
| Sphingosine-1-phosphate lyase 1 | 0 | 5 |
| Phosphatase and tenson homolog (PTEN) | −2.5 | 5 |
| Phosphatidic Acid Phosphate type 2a | −2.5 | 2.5 |
| Sphingomyelin phosphodiesterase 1 (acid | −5 | 0 |
| sphingomyelinase) | | |
| N-acylsphingosine amidohydrolase (acid | −15 | 2.5 |
| ceramidase) | | |
| EDG 2 | −17.5 | 0 |

TABLE 4

Lipid-Associate Gene Array Primers and Libraries

| | |
|---|---|
| Gene | Forward Primer |

| | | | | |
|---|---|---|---|---|
| Phosphatidylinositol-3-Kinase, catalytic, | 1213 | cgactttgcctttccatttgctc | 1235 | 2200 cctttt [symbol] |
| alpha polypeptide | | (SEQ ID NO. 32) | | (SEQ ID N [symbol] |
| Phosphocholine cytidyltransferase | 792 | aaaggagaaagtgaaagatgtggagg | 817 | 1186 ggac [symbol] |
| | | (SEQ ID NO. 34) | | (SEQ ID N [symbol] |
| Phospholipase D1 phosphatidylcholine | 1899 | ccccacttcaaactctttcaccc | 1921 | 2878 gcca [symbol] |
| specific | | (SEQ ID NO. 36) | | (SEQ ID N [symbol] |
| dihydroxyacetone phosphate acyltransferase | 900 | gctctgccaagacattgactcc | 921 | 2347 atcat [symbol] |
| | | (SEQ ID NO. 38) | | (SEQ ID N [symbol] |
| Phosphate cytidyltransferase 2, | 347 | cctacgtcactacactagagaccc | 370 | 600 gccaa [symbol] |
| ethanolamine specific | | (SEQ ID NO. 40) | | (SEQ ID N [symbol] |
| Phosphatidic Acid Phosphatase type 2c | 458 | aactgctcggtctatgtgcagc | 479 | 581 ccaag [symbol] |
| | | (SEQ ID NO. 42) | | (SEQ ID N [symbol] |
| Prostate differentiation factor PLAB | 881 | gctcattcaaaagaccgacaccg | 903 | 1154 acac [symbol] |
| (associated with Edg 3) | | (SEQ ID NO. 44) | | (SEQ ID N [symbol] |
| Phospholipase A2 | 474 | cgtctactgcctcaagagaaacc | 496 | 740 gtccta [symbol] |
| | | (SEQ ID NO. 46) | | (SEQ ID N [symbol] |
| Phospholipase C, beta 3 | 1137 | aggaagaggaggaacagacagac | 1159 | 1276 agca [symbol] |
| (phosphatidylinositol specific) | | (SEQ ID NO. 48) | | (SEQ ID N [symbol] |
| Phosphatidylinositol-3-Kinase, class 2, | 1713 | aacctgctgctgatagaccacc | 1734 | 3101 tctctc [symbol] |
| gamma polypeptide | | (SEQ ID NO. 50) | | (SEQ ID N [symbol] |
| Choline/ethanolamine phosphotransferase | 56 | gtaagcaccagccacaaaaacc | 77 358 | ctaacg [symbol] |
| | | (SEQ ID NO. 52) | | (SEQ ID N [symbol] |
| Lysophospholipase 1 | 119 | tggattgggagatactgggcac | 140 | 580 ccaaa [symbol] |
| | | (SEQ ID NO. 54) | | (SEQ ID N [symbol] |
| Aldehyde dehydrogenase 5 family, member | 930 | cctgttcttcaacatgggccag | 951 | 1308 cctct [symbol] |
| A1 | | (SEQ ID NO. 56) | | (SEQ ID N [symbol] |
| Phospholipase D1 | 1524 | tcttcttcccctaacatcaccatctc | 1549 | 2361 tgcat [symbol] |
| glycosyLphosphatidylinositol specific | | (SEQ ID NO. 58) | | (SEQ ID N [symbol] |
| 1-acylglycerol-3-phosphate 0- | 1043 | aaaccctcttccttgtctcccctc | 1066 | 1726 atgtc [symbol] |
| acyltransferase | | (SEQ ID NO. 60) | | (SEQ ID N [symbol] |
| Phosphatidic Acid Phosphatase type 2b | 358 | tcaacaacaacccgaggaggag | 379 | 710 gatggc [symbol] |
| | | (SEQ ID NO. 62) | | (SEQ ID N [symbol] |
| EDG 1 | 720 | acttccgcctcttcctgctaatc | 742 | 2009 cctcc [symbol] |
| | | (SEQ ID NO. 64) | | (SEQ ID N [symbol] |
| EDG 2 | 153 | atttcacagccccagttcacagcc | 176 | 633 tgacca [symbol] |
| | | (SEQ ID NO. 66) | | (SEQ ID N [symbol] |
| EDG 3 | 59 | agcattaccagtacgtggggaag | 81 | 356 aacata [symbol] |
| | | (SEQ ID NO. 68) | | (SEQ ID N [symbol] |
| EDG 4 | 797 | taggctgtgagtcctgcaatgtcc | 820 | 902 tcagca [symbol] |
| | | (SEQ ID NO. 70) | | (SEQ ID N [symbol] |
| EDG 5 | 28 | aaccccaacaaggtccaggaacac | 51 | 151 tttccac [symbol] |
| | | (SEQ ID NO. 72) | | (SEQ ID N [symbol] |
| EDG 6 | 1177 | aagttgcagtcttgcgtgtg | 1196 | 1382 ggtgg [symbol] |
| | | (SEQ ID NO. 74) | | (SEQ ID N [symbol] |
| EDG 7 | 378 | cttgactgcttccctcaccaac | 399 | 869 cttttcac [symbol] |
| | | (SEQ ID NO. 76) | | (SEQ ID N [symbol] |
| Sphingosine-1-phosphate lyase 1 | 797 | aggtggatgtgagggcaatgagaag | 821 | 1478 cgggc [symbol] |
| | | (SEQ ID NO. 78) | | (SEQ ID N [symbol] |
| Phosphatase and tenson homolog (PTEN) | 388 | gcctcctcttcgtcttttctaacc | 411 | 1631 catcat [symbol] |
| | | (SEQ ID NO. 80) | | (SEQ ID N [symbol] |
| Phosphatidic Acid Phosphatase type 2a | 186 | tcaaggcatacccccttccaac | 207 | 796 agtcca [symbol] |
| | | (SEQ ID NO. 82) | | (SEQ ID N [symbol] |
| Sphingomyelin phosphodiesterase 1 (acid | 716 | tctatgctctttccccatacccc | 738 | 1296 gcgat [symbol] |
| sphingomyelinase) | | (SEQ ID NO. 84) | | (SEQ ID N [symbol] |
| N-acylsphingosine amidohydrolase (acid | 279 | gtgccaagtggaaaagttatgcag | 302 | 1355 tgtcaa [symbol] |
| ceramidase | | (SEQ ID NO. 86) | | (SEQ ID N [symbol] |
| Glycerol-3-phosphate dehydrogenase | 836 | ccccatttatcagctccattgcc | 858 | 1562 catccc [symbol] |
| | | (SEQ ID NO. 88) | | (SEQ ID N [symbol] |
| Diacylglycerol kinase, gamma | 1360 | ccaacctactgcaacttctgcc | 1381 | 2446 caacc [symbol] |
| | | (SEQ ID NO. 90) | | (SEQ ID N [symbol] |
| Dihydroxyacetone phosphate | 900 | gctctgccaagacattgactcc | 921 | 2347 atcatc [symbol] |
| acyltransferase | | (SEQ ID NO. 92) | | (SEQ ID N [symbol] |
| Human Lysophospholipase Homolog | 936 | gttagccaagagccaggacaag | 957 | 1173 gcaag [symbol] |
| | | (SEQ ID NO. 94) | | (SEQ ID N [symbol] |

We claim:

1. A method for identifying tumor characteristics comprising:
measuring a copy number of at least two genes associated with lipid metabolism, synthesis, or action in cells of a patient tissue sample and comparing the results with a copy number in a normal cell.

2. The method of claim 1, wherein at least two of the genes associated with lipid metabolism, synthesis, or action are selected from the group consisting of: Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, Edg 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Triacylglycerol lipase, EDG 2, EDG 3, EDG 4, EDG 5, EDG 6, and EDG 7.

3. The method of claim 1, wherein at least one of the genes associated with lipid metabolism, synthesis, or action is selected from the group consisting of Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, Edg 1, Glycerol-3-phosphate dehydrogenase, EDG 2, EDG 3, EDG 4, EDG 5, EDG 6, and EDG 7.

4. The method of claim 1, wherein the determination of the copy number of the genes associated with lipid metabolism, synthesis, or action is made by:
isolating sample nucleic acid polymers from cells of the patient tissue sample;
hybridizing the sample nucleic acid polymers with nucleic acid polymers specific for the selected genes to quantify the extent of hybridization; and
comparing the hybridization data thus obtained with data obtained from the hybridization of reference nucleic acid polymers isolated from a normal cell of the same tissue type as the patient tissue sample.

5. The method of claim 4, wherein the sample nucleic acid polymer in the isolating step is amplified by a polymerase chain reaction (PCR).

6. The method of claim 4, wherein the step of hybridizing the sample nucleic acid polymers uses a nucleic acid polymer comprising at least about 19 nucleotides to hybridize to a coding region of one of the selected genes.

7. The method of claim 4, wherein the step of hybridizing the sample nucleic acid polymers uses a nucleic acid polymer comprising at least about 19 nucleotides to hybridize to a non-coding sequence functionally linked to the coding region of one of the selected genes, and wherein the non-coding functionally linked sequence is unique to that gene.

8. The method of claim 4, wherein the hybridizing step uses nucleic acid polymers specific for the selected genes that are immobilized on a solid support wherein a nucleic acid polymer specific for each selected gene is located at a predetermined position on the solid support.

9. A method for identifying tumor characteristics comprising measuring the expression level of at least two genes associated with lipid metabolism, synthesis, or action in a patient tissue sample and comparing the results with an expression level in normal cells.

10. The method of claim 9, wherein at least two of the genes associated with lipid metabolism, synthesis, or action are selected from the group consisting of:
Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, Edg 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Triacylglycerol lipase, EDG 2, EDG 3, EDG 4, EDG 5, EDG 6, and EDG 7.

11. The method of claim 9, wherein at least one of the genes associated with lipid metabolism, synthesis, or action is selected from the group consisting of Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, Edg 1, Glycerol-3-phosphate dehydrogenase, EDG 2, EDG 3, EDG 4, EDG 5, EDG 6, and EDG 7.

12. The method of claim 9, wherein the determination of the expression level of the genes associated with lipid metabolism, synthesis, or action is made by:
isolating sample ribonucleic acid polymers from the cells of the patient tissue sample;
hybridizing the sample ribonucleic acid polymers with nucleic acid polymers specific for the selected genes to quantify the extent of hybridization; and
comparing the hybridization data thus obtained with data obtained from the hybridization of reference nucleic acid polymers isolated from a normal cell of the same tissue type as the patient tissue sample.

13. The method of claim 12, wherein the sample ribonucleic acid polymer in the isolating step is amplified by a polymerase chain reaction (PCR) technique.

14. The method of claim 12, wherein the step of hybridizing the sample ribonucleic acid polymers uses a nucleic acid polymer comprising at least about 19 nucleotides to hybridize to a coding region of one of the selected genes.

15. The method of claim 12, wherein the step of hybridizing the sample ribonucleic acid polymers uses a nucleic acid polymer comprising at least about 19 nucleotides to hybridize to a non-coding sequence functionally linked to the coding region of one of the selected genes, wherein the functionally linked non-coding sequence is unique to that gene.

16. The method of claim 12, wherein the hybridizing step uses nucleic acid polymers specific for the selected genes that are immobilized on a solid support wherein a nucleic acid polymer specific for each selected gene is located at a predetermined position on the solid support.

17. A physical platform comprising an array of nucleic acid polymers immobilized at a predetermined position on a solid support:
wherein the array is comprised of at least two different isolated nucleic acid polymers which are each specific for a different gene associated with lipid metabolism, synthesis, or action; and
genomic DNA derived from a patient tissue sample that is further comprised of a label and that contacts the array under conditions wherein hybridization of the genomic DNA and the immobilized nucleic acid polymers are determined by detecting the label at the predetermined position of at the at least two isolated nucleic acid polymers.

18. The platform of claim 17 wherein the at least two isolated nucleic acid polymers are specific for two genes selected from the group consisting of Phosphatidylinositol-3-kinase (catalytic, alpha polypeptide), Phospholipase D1 (phosphatidylcholine specific), Dihydroxyacetone phosphate acyltransferase, Phosphate cytidylyltransferase 1 (choline specific, alpha form), Phosphate cytidylyltransferase 2 (ethanolamine specific), Phosphatidic Acid Phosphatase type 2c, Prostate Differentiation Factor PLAB, Phospholipase A2, Phospholipase C beta 3 (phosphatidylinositol specific), Phosphatidylinositol-3-Kinase (class2, gamma polypeptide), Choline/ethanolamine phosphotransferase, Lyosphospholipase, Aldehyde dehydrogenase (5 family, member A1), Phospholipase D1 glycosylphosphatidylinositol specific, 1-acylglycerol-3-phosphate acyltransferase, Phosphatidic Acid Phosphate type 2b, Edg 1, Glycerol-3-phosphate dehydrogenase, Sphingosine-1-phosphate lyase 1, Phosphatase and Tenson Homolog (PTEN), Phosphatidic Acid Phosphatase type 2a, Sphingomyelin phosphodiesterase 1, N-acylsphingosine amidohydrolase, Glycerol Kinase, Diacylglycerol Kinase gamma, Acyl-dihydroxyacetone phosphate reductase, Triacylglycerol lipase, EDG 2, EDG 3, EDG 4, EDG 5, EDG 6, and EDG 7.

19. The platform of claim 18 wherein at least one of the at least two genes is selected from the group consisting of Phosphatidylinositol-3-kinase (catalytic, alpha polypeptide), Phospholipase D1 (phosphatidylcholine specific), Prostate Differentiation Factor PLAB, Phospholipase A2, Phospholipase D1 glycosylphosphatidylinositol specific, Edg 1, Glycerol-3-phosphate dehydrogenase, EDG 2, EDG 3, EDG 4, EDG 5, EDG 6, and EDG 7.

20. The platform of claim 17 wherein at least one of the isolated nucleic acid polymers is comprised of at least about 19 nucleotides.

21. The platform of claim 17 wherein at least one of the isolated nucleic acid polymers specific for the selected genes is a nucleic acid polymer comprising at least about 19 nucleotides which hybridize under the conditions to a non-coding sequence functionally linked to the coding region of one of the selected genes, wherein the functionally linked sequence is unique to that gene.

22. A physical platform, comprising:
an array of nucleic acid polymers immobilized on a solid support,
wherein the array is comprised of at least two different isolated nucleic acid polymers which are each specific for a different gene directly associated with lipid metabolism, synthesis, or action.

23. A physical platform, comprising:
an array of nucleic acid polymers immobilized on a solid support,
wherein the array is comprised of at least two different isolated nucleic acid polymers which are each specific for a different gene encoding a protein that modifies, oxidizes, reduces, cleaves, binds, or otherwise utilizes bioactive lipids as substrates or ligands.
